(19) United States
(12) Patent Application Publication
(10) Pub. No.: US 2025/0152753 A1
(43) Pub. Date: May 15, 2025

(54) FORMULATION OF CONTRAST MEDIA AND PROCESS OF PREPARATION THEREOF

(71) Applicants: BAYER AKTIENGESELLSCHAFT, Leverkusen (DE); BAYER PHARMA AKTIENGESELLSCHAFT, Berlin (DE)

(72) Inventors: Stephan Holzschuh, Haan (DE); Thomas Frenzel, Berlin (DE); Gregor Jost, Berlin (DE); Jessica Lohrke, Berlin (DE); Wolfgang Ebert, Krefeld (DE); Thomas Brumby, Berlin (DE); Wolfgang Halfbrodt, Berlin (DE)

(21) Appl. No.: 19/025,116

(22) Filed: Jan. 16, 2025

Related U.S. Application Data
(63) Continuation of application No. 18/617,245, filed on Mar. 26, 2024.
Application No. 18/617,245 is a continuation of application No. 17/295,647, filed on May 20, 2021, now Pat. No. 11,944,690.

(30) Foreign Application Priority Data
Nov. 23, 2018 (EP) 18208090.3

(51) Int. Cl. A61K 49/10; A61K 49/08; A61K 49/12

(52) U.S. Cl. CPC A61K 49/108; A61K 49/085; A61K 49/124

(57) ABSTRACT

The present disclosure relates to a liquid pharmaceutical formulation comprising a DO3A-derived tetra-chelate of formula (I), in which M is an ion of a paramagnetic metal, preferably a Gd³⁺ ion, and R¹, R², R³, R⁴ and R⁵ are as defined in the claims, in a pharmaceutical acceptable solvent. The present disclosure also relates to a method of preparation of said liquid pharmaceutical formulation and to a method of imaging involving said liquid pharmaceutical formulation.

### CROSS REFERENCE TO RELATED APPLICATIONS

The present application is a continuation of U.S. Ser. No. 18/617,245, filed 26 Mar. 2024, which is a continuation of U.S. Ser. No. 17/295,647, filed 20 May 2021, which issued as U.S. Pat. No. 11,944,690 on 2 Apr. 2024, which was a U.S. national stage filing under 35 U.S.C. § 371 of International Application No. PCT/EP2019/082117, filed 21 Nov. 2019, which claims priority to European Patent Application No. EP 18208090.3, filed 23 Nov. 2018, the disclosures of each of which are incorporated in their entirety herein by this reference.

### FIELD OF THE DISCLOSURE

The present invention relates to formulations of contrast agents, in particular of paramagnetic metal ion chelates, in particular for magnetic resonance imaging, and to industrially applicable processes for obtaining said formulations.

### BACKGROUND

Various contrast agents based on lanthanide (paramagnetic metal) chelates, in particular gadolinium chelates, described for example in document U.S. Pat. No. 4,647,447, are known.

These products are termed GBCAs (Gadolinium-based Contrast Agents). Several GBCAs have been approved for clinical use and are commercially available: in particular linear Gd-chelates, such as gadopentetate dimeglumine (Magnevist®, based on DTPA), gadodiamide (Omniscan®, based on DTPA-BMA), gadoversetamide (OptiMARK®, based on DTPA-BMEA) and in particular macrocyclic Gd-chelates, gadoterate meglumine (Dotarem®, based on DOTA), gadoteridol (ProHance®, based on HP-DO3A), gadobutrol (Gadovist®, based on BT-DO3A, Butrol), gadoxetic acid (Primovist®, based on EOB-DTPA) and gadobenate dimeglumine (MultiHance®, based on BOPTA).

These compounds will, in the remainder of the text, be referred to without distinction as “Gd-chelates” or “chelates” and their ligands as “chelating ligands”.

Some GBCAs are described in the following documents U.S. Pat. Nos. 6,440,956, 5,403,572, EP 0 438 206, WO 93/011800.

Magnetic resonance imaging (MRI) contrast agents may be characterized by their longitudinal (r₁) and transverse (r₂) relaxivities. Relaxivity is the degree to which the agent can enhance the longitudinal or transverse water relaxation rate constant (R₁=1/T, or R₂=1/T₂, respectively) normalized to the concentration of the contrast agent. Relaxivity is a measure of the efficacy of the contrast agent (Jacques V. et al. Invest. Radiol. 2010 October; 45(10): 613-624). The various GBCAs differ, for example, in their relaxivities which are dependent on factors such as magnetic field strengths, temperature, and different intrinsic factors of the metal chelates. The intrinsic relaxivity influencing parameters are mainly the number of water molecules directly bound to the gadolinium (so-called inner-sphere water, q), the mean residence time of the inner sphere water molecules (τm), the number and residence times of water molecules in the second hydration sphere (so-called second sphere water), and the rotational diffusion (τr) (Helm L. et. al., Future Med. Chem. 2010; 2: 385-396). In terms of their relaxivity, commercially available GBCAs are similar to each other and falling within a range of 4 to 7 L mmol⁻¹ s⁻¹.

A further characteristic of GBCAs is the complex stability of the Gd-chelate.

In certain GBCAs, small amounts of free gadolinium ions may be released after administration to the patient or decomplexation may occur during storage/shipping. This has led to a search for technical solutions to limit free metal ion exposure in order to safely solve the complex technical problem of tolerance in the patient. For example since 2006, a pathological condition known as NSF (Nephrogenic Systemic Fibrosis) has been at least partly linked to the administration of GBCAs and subsequent presence of gadolinium in the body. This disease has resulted in a warning by health authorities with respect to specific GBCAs used in patients with reduced or no kidney function. Another example is the accumulation of gadolinium in the brain, which has been observed after multiple administration of certain linear GBCAs. As the administration of contrast agents is often repeated during the patient's treatment cycle for guiding and monitoring the effectiveness of a therapeutic treatment, the risk of patient exposure to the free gadolinium ions increases.

The complex problem of the tolerance of new GBCAs must always be considered.

As described herein, development of new high relaxivity contrast agents with higher efficacy can lead to a significant reduction in the administered dose and thus reduces the risk of accumulation of Gd in the body.

Another strategy for limiting this risk is the selection of lanthanide chelates which have the highest possible thermodynamic stabilities and kinetic inertness. This is because the higher the stability and inertness of the chelate, the amount of lanthanide released over time is reduced.

Several strategies for improving Gd-chelate tolerance are described, for example, in U.S. Pat. No. 5,876,695 and WO 2009/103744, which disclose formulations comprising an excess of free chelating ligand, intended to inhibit an unwanted accumulation of gadolinium, by complexing any released gadolinium. U.S. Pat. No. 5,876,695 describes an excess of linear chelating ligand, in particular of free DTPA. This formulation strategy has been used for commercial products such as Magnevist®. WO 2009/103744 describes a similar formulation strategy, based on the addition of free chelating ligand, in particular of free DOTA, so as to have a very small excess of said free chelating ligand to complex any released metal, resulting in a zero concentration of free gadolinium. However, certain chelating ligands may also have a toxicity profile that limits the amount of free ligand that can be added to the formulation.

US 2004/0170566, EP 0 454 078 and U.S. Pat. No. 5,876,695 describe formulations containing “weak” complexes or salts of chelating ligands and transition metals or alkaline-earth metals with much lower thermodynamic stability than the corresponding Gd-chelate. These “weak” complexes (e.g. Ca-, Zn-, Na- or Mg-complexes) undergo transmetallation in the presence of free lanthanide as they are thermodynamically more stable.

US 2016/0101196A1 describes a formulation composition comprising a PCTA derived mono-Gd-complex and also comprising a calcium complex of 1,4,7,10-tetraazacyclododecane-1,4,7,10-tetraacetic acid (Ca-DOTA).

### BRIEF DESCRIPTION

The Applicant has carried out studies on the specific case of the DO3A-derived tetra-chelates as described in WO 2016/193190 and WO 2018/096082. The strategies described above have been developed only for monomeric Gd-chelates and not for the DO3A-derived tetra-chelates as described herein.

It has now been found, as described herein, that the various embodiments of the formulations of the present disclosure have surprising and advantageous properties.

Pharmaceutical formulations described herein which comprise DO3A-derived tetra-chelates of lanthanide ions, disclosed in WO 2016/193190 and WO 2018/096082, displaying high relaxivity as well as other useful properties for use as a contrast agent in medical imaging procedures, for example in magnetic resonance imaging (MRI) procedures are described.

According to one embodiment, formulations comprising small amounts of a lanthanide ion scavenging agent, such as a scavenging agent comprising a chelating ligand that forms a strong complex with the free lanthanide ion, are described. The scavenging agent may comprise the free form of the chelating ligand (i.e., the ligand in an uncomplexed form) and/or the ligand as a complex with a weakly binding metal ion, such as ions of the alkali metals or the alkaline earth metals or weakly binding transition metal ions.

According to certain embodiments, the Applicant has discovered that the addition of small amounts of the calcium complex of 10-[2,3-dihydroxy-1-(hydroxymethyl)propyl]-1,4,7,10-tetraazacyclododecane-1,4,7-triacetic acid (Ca-BT-DO3A, Calcobutrol) to a formulation comprising a DO3A-derived tetra-chelate described herein, such as a Gd₄-DO3A-derived tetra-chelate, ensures the absence of free paramagnetic metal ion in the formulation, in particular in the injectable solution, while preserving the performance levels as a contrast medium in medical imaging procedures.

According to other embodiments, similar effects may be achieved, when the formulation comprises a poly-chelate, which is a chelate having more than one metal chelating site, i.e. having 2-64 metal chelating sites, and includes amounts of ligand chelate with sub-stoichiometric amounts of paramagnetic metal ions which provide sufficient chelating activity to bind any free paramagnetic metal ions in the formulation.

According to other embodiments, similar effects may be achieved, when the formulation comprises a DO3A-derived tetra-chelate, such as a Gd₄-DO3A-derived tetra-chelate, and includes amounts of ligand chelate with sub-stoichiometric amounts of paramagnetic metal ions (i.e. 0, 1, 2 or 3 paramagnetic metal ions) which provide sufficient chelating activity to bind any free paramagnetic metal ions in the formulation. In other embodiments, the amount of ligand chelate with sub-stoichiometric amounts of paramagnetic metal ion may have a corresponding amount of one to four weakly binding metal ions, as described herein.

The pharmaceutical formulations of certain embodiments have a concentration of free paramagnetic metal of less than or equal to 5 ppm (m/v), i.e. in the range of 0 to 5 ppm (m/v) (inclusive), and in certain embodiments less than or equal to 2 ppm (m/v), i.e. in the range of 0 to 2 ppm (m/v) (inclusive), and in certain embodiments less than or equal to 0.5 ppm (m/v), i.e. in the range of 0 to 0.5 ppm (m/v) (inclusive).

According to various embodiments, the viscosity of the pharmaceutical formulations of the current disclosure has been found to be only slightly higher than for isotonic sodium chloride solution. According to certain embodiments, the pharmaceutical formulations described herein may have a lower viscosity compared to conventional contrast formulations. The low viscosity leads to a good local tolerance of the intravenous bolus application and allows a convenient and reproducible application through a long and thin catheter during hand injection (less pressure required) and/or avoids fluid flow rate changes during fluid transitions from contrast to saline injection during injections with powered injection systems.

In contrast to the available market products, in several embodiments of the pharmaceutical formulation of the present disclosure, the formulation is isotonic with blood plasma, which also increases the tolerability at the site of injection when compared with the hyperosmolar formulations of commercially available imaging contrast formulations.

### DETAILED DESCRIPTION

One subject of the present disclosure therefor relates to a liquid pharmaceutical formulation comprising a DO3A-derived tetra-chelate.

Another subject of the present disclosure relates to a liquid pharmaceutical formulation comprising a DO3A-derived tetra-chelate, and further comprising one or more scavenging agents, which are defined as compounds capable of forming a complex with free paramagnetic metal ion M.

Another subject of the present disclosure relates to a liquid pharmaceutical formulation comprising a DO3A-derived tetra-chelate and a calcium complex of 10-[2,3-dihydroxy-1-(hydroxymethyl)propyl]-1,4,7,10-tetraazacyclododecane-1,4,7-triacetic acid (Ca-BT-DO3A, Calcobutrol).

Another subject of the present disclosure relates to a liquid pharmaceutical formulation comprising a DO3A-derived tetra-chelate and DO3A-derived tetra-chelates with sub-stoichiometric amounts of paramagnetic metal ions.

Another subject of the disclosure relates to a contrast medium for medical imaging, such as magnetic resonance imaging, comprising said liquid pharmaceutical formulation.

Another subject of the present disclosure relates to a process for preparing said liquid pharmaceutical formulation.

The present disclosure also relates to said liquid pharmaceutical formulation or said contrast medium for use thereof in a diagnostic method, such as a magnetic resonance imaging diagnostic method.

As described herein, a chelate of general formula (I), which is a chelate between a chelating ligand of general formula (II) and four paramagnetic metal ions, will be referred to as “DO3A-derived tetra-chelate”. More specifically and unless otherwise indicated, the complex between the chelating ligand of general formula (II) and gadolinium (Gd³⁺) ions will be referred to as “Gd₄-DO3A-derived tetra-chelate”.

The chelating ligand of general formula (II) is the free ligand (i.e., no complexed metal ion) and will be referred to as “DO3A-derived tetra-ligand”.

DO3A-derived tetra-chelates with sub-stoichiometric amounts of paramagnetic metal ions are compositions containing DO3A-derived tetra-chelates and further including one or more chelates between a DO3A-derived tetra-ligand of general formula (II) and sub-stoichiometric amounts of paramagnetic metal ions, such as one, two or three paramagnetic metal ions, and/or containing the DO3A-derived tetra-ligand of general formula (II), or mixtures thereof. In various embodiments, the DO3A-derived tetra-ligand comprising sub-stoichiometric amounts of paramagnetic metal ions may include sub-stoichiometric amounts of one or more weak binding metal ions. As used herein, the term “weak binding metal ions” include metal ions for the alkali metals, alkaline earth metals, and transition metals that have a binding affinity for the DO3A-derived tetra-chelate that is less than the binding affinity of the DO3A-derived tetra-chelate and the lanthanide metal ion. In various embodiments, the weak binding metal ions may include lithium, calcium, sodium, zinc, potassium, or magnesium ions.

Gd₄-DO3A-derived tetra-chelates with sub-stoichiometric amounts of paramagnetic gadolinium ions are compositions containing Gd₄-DO3A-derived tetra-chelates and further including chelates between a DO3A-derived tetra-ligand of general formula (II) and one, two or three Gd³⁺ ions, and/or containing the DO3A-derived tetra-ligand of general formula (II) as a free ligand, or mixtures thereof. In various embodiments, the Gd₄-DO3A-derived tetra-ligand comprising sub-stoichiometric amounts of paramagnetic gadolinium ions and/or the free DO3A-derived tetra-ligand may include sub-stoichiometric or stoichiometric amounts, respectively, of one or more weak binding metal ions in the gadolinium-free ligating sites.

A sub-stoichiometric chelate between a DO3A-derived tetra-ligand of general formula (II) and three paramagnetic metal ions will be referred to as “M₃-DO3A-derived chelate”. A sub-stoichiometric chelate between a DO3A-derived tetra-ligand of general formula (II) and two paramagnetic metal ions will be referred to as “M₂-DO3A-derived chelate”. A sub-stoichiometric chelate between a DO3A-derived tetra-ligand of general formula (II) and one paramagnetic metal ion will be referred to as “M-DO3A-derived chelate”. According to various embodiments, these sub-stoichiometric chelates as well as the DO3A-derived tetra-ligand of general formula (II), which also is a sub-stoichiometric chelate, can be present in the formulation.

More specifically and unless otherwise indicated, a sub-stoichiometric chelate between a DO3A-derived tetra-ligand of general formula (II) and three Gd³⁺ ions will be referred to as “Gd₃-DO3A-derived chelate”, a sub-stoichiometric chelate between a DO3A-derived tetra-ligand of general formula (II) and two Gd³⁺ ions will be referred to as “Gd₂-DO3A-derived chelate”, and a sub-stoichiometric chelate between a DO3A-derived tetra-ligand of general formula (II) and one Gd³⁺ ion will be referred to as “Gd-DO3A-derived chelate”.

In general, one aspect of the present disclosure includes pharmaceutical formulations of poly-ligands of paramagnetic metal ions, such as lanthanide metal ions, having two or more metal chelating sites, such as from 2-64 metal chelating sites, wherein each of the metal chelating sites has a paramagnetic metal ion bound thereto, wherein the formulation further comprises amounts of the poly-ligand having a sub-stoichiometric amount of the paramagnetic metal ions bound thereto.

According to these embodiments, the chelating sites of the poly-ligand having sub-stoichiometric amounts of paramagnetic metal ions may be free (i.e., no bound metal) or may have a weak metal ion bound thereto. Further, according to these embodiments, one or more of the chelating sites of the poly-ligand having sub-stoichiometric amounts of paramagnetic metal ions may be free (i.e., no bound metal) or may have a weak metal ion bound thereto.

Pharmaceutical formulations such as these will have incorporated therein a metal scavenging moiety that is capable of binding to any released paramagnetic metal ion that may be released during storage, shipping, or during an injection protocol, thereby preventing its release into a patient's blood stream or organs.

The complex between 10-[2,3-dihydroxy-1-(hydroxymethyl)propyl]-1,4,7,10-tetraazacyclo-dodecane-1,4,7-triacetic acid (Butrol) and calcium ions, which is known as Calcobutrol, will be referred to as “Ca-BT-DO3A”.

In accordance with a first aspect, the present disclosure covers a liquid pharmaceutical formulation comprising a DO3A-derived tetra-chelate of general formula (I):

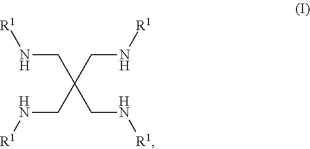

in which
R¹ represents a group selected from:

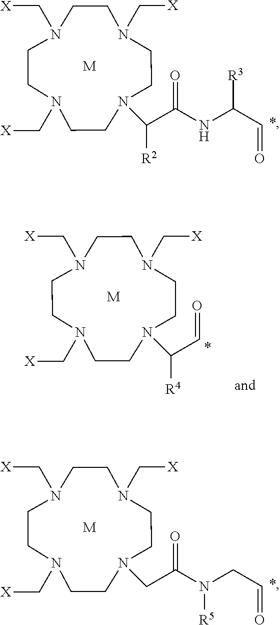

in which group * indicates the point of attachment of said group with the rest of the molecule,
R², R³, and R⁴ independently of each other represents a hydrogen atom or a group selected from: C₁-C₆-alkyl, C₃-C₆-cycloalkyl, (C₁-C₂-alkoxy)-(C₂-C₃-alkyl)-, and phenyl,
   wherein said C₁-C₆-alkyl group is optionally substituted, identically or differently, with a phenyl substituent, which phenyl substituent is optionally substituted, one, two or three times, identically or differently, with a halogen atom or a group selected from:
      C₁-C₃-alkyl, C₁-C₃-haloalkyl, and C₁-C₃-alkoxy, and wherein said phenyl group is optionally substituted, one, two or three times, identically or differently, with a halogen atom or a group selected from:
      C₁-C₃-alkyl, C₁-C₃-haloalkyl, and C₁-C₃-alkoxy,
R⁵ represents a group selected from:
   C₁-C₆-alkyl, C₃-C₆-cycloalkyl, (C₁-C₂-alkoxy)-(C₂-C₃-alkyl)- and phenyl, wherein said C₁-C₆-alkyl group is optionally substituted, identically or differently, with a phenyl substituent, which phenyl substituent is optionally substituted, one, two or three times, identically or differently, with a halogen atom or a group selected from:
      C₁-C₃-alkyl, C₁-C₃-haloalkyl, and C₁-C₃-alkoxy, and wherein said phenyl group is optionally substituted, one, two, or three times, identically or differently, with a halogen atom or a group selected from:
      C₁-C₃-alkyl, C₁-C₃-haloalkyl, and C₁-C₃-alkoxy,
X represents a group C(═O)OH or C(═O)O—,
and
M represents an ion of a paramagnetic metal,
or a stereoisomer, a tautomer or a salt thereof, or a mixture of same,
said formulation comprising
   a pharmaceutically acceptable solvent,
   and optionally comprising a buffer,
wherein the DO3A-derived tetra-chelate has a concentration in the formulation in a range of 1 to 1000 mmol paramagnetic metal ion/L (inclusive), wherein the ion of the paramagnetic metal is not Gd³⁺, and in a range of 60 to 750 mmol paramagnetic metal ion/L (inclusive) wherein the ion of the paramagnetic metal can also be Gd³⁺, particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion ion/L (inclusive), more particularly in a range of 200 to 400 mmol paramagnetic metal ion/L (inclusive), and even more particularly in a range of 250 to 350 mmol paramagnetic metal ion/L (inclusive).

### Definitions

All concentrations or dosings references related to the various contrast formulations of this disclosure, unless otherwise noted, refer to concentration of the paramagnetic metal ion. This is important, because the tetrameric complexes carry 4 paramagnetic metal ions per molecule.

Thus, a formulation comprising Gd₄-DO3A-derived tetra-chelate having a 1 mmol/L concentration of the ligand/metal chelate would have a concentration of 4 mmol/L of the Gd³⁺ ion.

The terms “formulation” and “pharmaceutical formulation” mean, according to the disclosure, a solution comprising at least a DO3A-derived tetra-chelate of formula (I), supra, in a “pharmaceutically acceptable solvent”, wherein the term “pharmaceutically acceptable solvent” is intended to mean a solvent that is suitable with parenteral application, i.e., water, an aqueous solution, or one or more compounds selected from the list of solvents herein, that is capable of substantially solubilizing the DO3A-derived tetra-chelate, which solution optionally comprises further one or more pharmaceutically suitable excipients.

Pharmaceutically suitable excipients include, inter alia,
solvents (for example water, ethanol, isopropanol, glycerol, propylene glycol and liquid polyethylene glycols),
buffers, acids, and/or bases (for example buffers comprising phosphates, carbonates, citrate, ascorbates, acetate, succinate, malate, maleate, lactate, tartrate, trometamol (TRIS, 2-amino-2-(hydroxymethyl)propane-1,3-diol), triethanolamine, HEPES (2-[4-(2-hydroxyethyl)-1-piperazine]ethanesulfonic acid), MES (2-morpholinoethanesulfonic acid), and sodium hydroxide, and hydrochloric acid),
isotonicity agents (for example glucose, sodium chloride, d-mannitol, sorbitol, sucrose, trehalose, and propylene glycol),
stabilisers (for example antioxidants such as, for example, ascorbic acid, ascorbyl palmitate, and sodium ascorbate),
preservatives (for example sorbic acid).

The term “buffer solution” means, according to the disclosure, a solution in a pharmaceutically acceptable solvent which comprises a buffer, which buffer is chosen from citrate, lactate, acetate, tartrate, malate, maleate, phosphate, succinate, ascorbate, carbonate, trometamol s, HEPES (2-[4-(2-hydroxyethyl)-1-piperazine]ethanesulfonic acid) and MES (2-morpholinoethanesulfonic acid) and mixtures of any thereof.

The term “substituted” means that one or more hydrogen atoms on the designated atom or group are replaced with a selection from the indicated group, provided that the designated atoms normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing any hydrogen atom with a non-hydrogen substituent on any available carbon atom.

Should a composite substituent be composed of more than one part, e.g.

(C₁-C₂-alkoxy)-(C₂-C₃-alkyl)-, it is possible for a given part to be attached at any suitable position of said composite substituent, e.g. it is possible for the C₁-C₂-alkoxy part to be attached to any suitable carbon atom of the C₂-C₃-alkyl part of said

(C₁-C₂-alkoxy)-(C₂-C₃-alkyl)- group. A hyphen at the beginning or at the end of such a composite substituent indicates the point of attachment of said composite substituent to the rest of the molecule.

The term “comprising” when used in the specification includes “consisting essentially of” and “consisting of”.

The terms as mentioned in the present text have the following meanings:

The term “halogen atom” means a fluorine, chlorine, bromine or iodine atom, particularly a fluorine, chlorine, or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “C₁-C₃-alkyl” means a linear or branched, saturated, monovalent hydrocarbon group having 1, 2 or 3 carbon atoms, e.g., a methyl, ethyl, propyl, or isopropyl group.

The term “C₂-C₃-alkyl” means a linear or branched, saturated, monovalent hydrocarbon group having 2 or 3 carbon atoms, e.g., an ethyl, propyl, or isopropyl group.

The term “C₁-C₂-alkyl” means a linear, saturated, monovalent hydrocarbon group having 1 or 2 carbon atoms, e.g., a methyl or ethyl group.

The term “C₃-C₆-cycloalkyl” means a saturated, monovalent, monocyclic hydrocarbon ring which contains 3, 4, 5, or 6 carbon atoms. Said C₃-C₆-cycloalkyl group is, for example, a cyclopropyl, cyclobutyl, cyclopentyl, or cyclohexyl group.

The term “C₁-C₃-haloalkyl” means a linear or branched, saturated, monovalent hydrocarbon group in which the term “C₁-C₃-alkyl” is as defined supra, and in which one or more of the hydrogen atoms are replaced, identically or differently, with a halogen atom as defined supra.

Particularly, said halogen atom is a fluorine atom. Said C₁-C₃-haloalkyl group is, for example, fluoromethyl, difluoromethyl, trifluoromethyl, 2-fluoroethyl, 2,2-difluoroethyl, 2,2,2-trifluoroethyl, pentafluoroethyl, 3,3,3-trifluoropropyl, or 1,3-difluoropropan-2-yl.

The term “C₁-C₃-alkoxy” means a linear or branched, saturated, monovalent group of formula (C1-C₃-alkyl)-O—, in which the term “C₁-C₃-alkyl” is as defined supra, e.g., a methoxy, ethoxy, n-propoxy, or isopropoxy group.

The term “C₁-C₂-alkoxy” means a linear saturated, monovalent group of formula (C₁-C₂-alkyl)-O—, in which the term “C₁-C₂-alkyl” is as defined supra, e.g., a methoxy or ethoxy group.

When a range of values is given, said range encompasses each terminal value and any sub-range within said range.

For example:
“C₁-C” encompasses C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;
“C₁-C₄” encompasses C₁, C₂, C₃, C₄, C₁-C₄, C₁-C₃, C₁-C₂, C₂-C₄, C₂-C₃, and C₃-C₄;
“C₁-C₃” encompasses C₁, C₂, C₃, C₁-C₃, C₁-C₂, and C₂-C₃;
“C₃-C” encompasses C₃, C₄, C₅, C₆, C₃-C₅, C₃-C₆, C₃-C₄, C₄-C₅, C₄-C₆, and C₅-C₆.

It is possible for the compounds of general formulae (I) and (II) to exist as isotopic variants. The disclosure therefor may also include one or more isotopic variant(s) of the compounds of general formulae (I), (I-a), (I-b), (I-c), (II), (II-a), (II-b) and (II-c), and of any of the compounds having sub-stoichiometric amounts of paramagnetic metal ion, as described herein, such as deuterium-containing compounds of general formulae (I), (I-a), (I-b), (I-c), (II), (II-a), (II-b) and (II-c), and of any of the compounds having sub-stoichiometric amounts of paramagnetic metal ion, as described herein, where one or more ¹H atoms has been replaced with a ²H atom.

The term “isotopic variant” of a compound is defined as a compound exhibiting an unnatural proportion of one or more of the isotopes that constitute such a compound.

The term “isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

It is possible for the compounds of general formula (I) to exist as stereochemical variants. The disclosure therefor also covers all stereochemical variants or combinations of compounds possessing one or more chiral centers as described herein, such as, compounds of general formulae (I), (I-a), (II), and (II-a), and any of the compounds having sub-stoichiometric amounts of paramagnetic metal ion, as described herein. The term “stereochemical variant” means that any chiral carbon center in the compound may exist in either the R or S stereochemical format and that compounds having more than one chiral carbon center may exist as any combination of enantiomers and diastereomers where the chiral centers may be any combination having either R or S stereochemistry at each chiral carbon center. The compound of general formula (I), (I-a), (II), and (II-a), and the compounds having sub-stoichiometric amounts of paramagnetic metal ion, i.e. the compounds of general formula (Gd₃-II-a), (Gd₂-II-a), (Gd-II-a), as described herein, may exist in enantiomerically or diastereomerically pure form, may be a mixture of enantiomers (such as a racemic mixture of enantiomers or a mixture that is enantiomerically enriched in the amount of one enantiomer relative to the other enantiomer), may be a mixture of diastereomers (such as a random mixture of two or more diastereoisomers or a mixture of two or more diastereoisomers wherein the mixture is enriched in the amount of one or more diastereoisomer relative to the amount of the one or more other diastereoisomers in the mixture).

The term “sub-stoichiometric amount” means less than the stoichiometric amount. When used in reference to a chelate having one or more chelating sites for a metal ion, sub-stoichiometric amounts of a metal ion means a molar amount of the metal ion that is less than the molar amount of available chelating sites capable of chelating to a metal ion.

The term “sub-stoichiometric chelate” means a chelate between a ligand and a sub-stoichiometric number of ions of a paramagnetic metal, i.e. a chelate between a tetra-ligand and 0, 1, 2 or 3 ions of a paramagnetic metal.

In accordance with a second embodiment of the first aspect, the present disclosure covers a liquid pharmaceutical formulation, supra, comprising a DO3A-derived tetra-chelate of general formula (I), supra, in which:
R² represents a hydrogen atom or a methyl group,
R³ and R⁴ each represent a hydrogen atom, and
R⁵ represents a group selected from:
   methyl, ethyl, isopropyl, 2-methylpropyl, benzyl, cyclopropyl, cyclopentyl, cyclohexyl, 2-methoxyethyl, 2-ethoxyethyl, and phenyl.

In accordance with a third embodiment of the first aspect, the present disclosure covers a liquid pharmaceutical formulation, supra, wherein the DO3A-derived tetra-chelate of formula (I) is selected from the chelates of formulae (I-a), (I-b), and (I-c) having structures as follows:

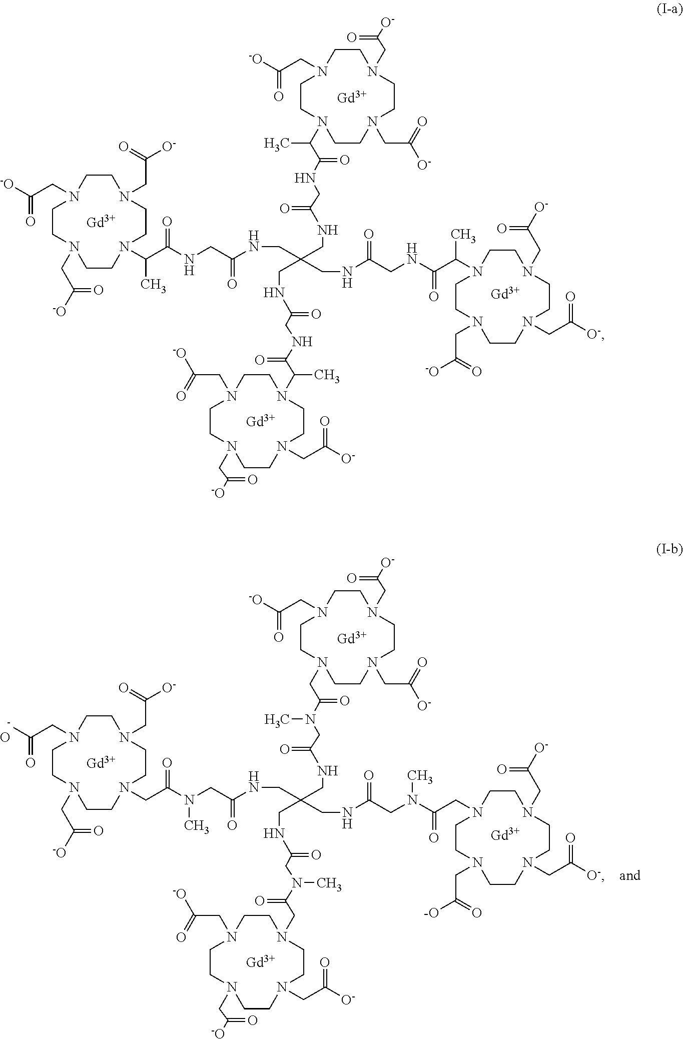

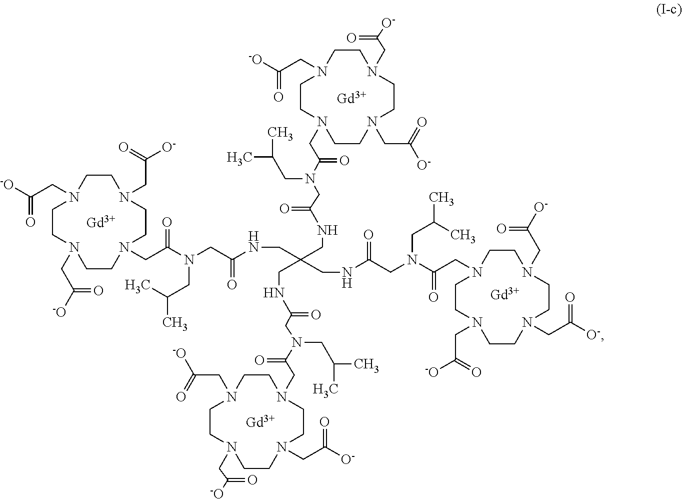

or a stereoisomer, a tautomer or a salt thereof, or a mixture of same.

In accordance with a fourth embodiment of the first aspect, the present disclosure covers a liquid pharmaceutical formulation, supra, wherein the DO3A-derived tetra-chelate of formula (I) has the formula (T-a) as follows:

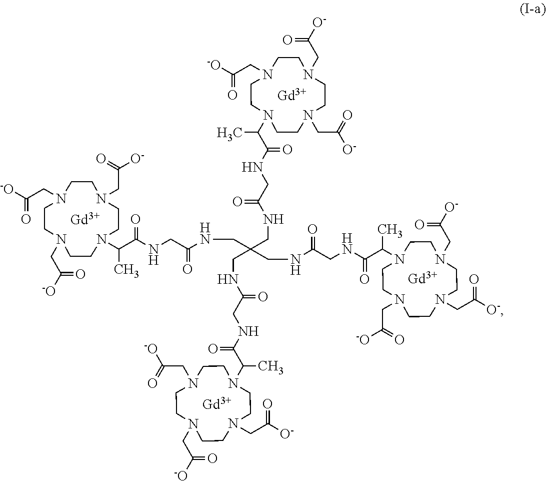

or a stereoisomer, a tautomer or a salt thereof, or a mixture of same.

In accordance with an fifth embodiment of the first aspect, the formulation according to the disclosure has a concentration of the Gd₄-DO3A-derived tetra-chelate of general formula (I), supra, in a range of 60 to 750 mmol Gd³⁺/L (inclusive), particularly in a range of 70 to 700 mmol Gd³⁺/L (inclusive), particularly in a range of 80 to 650 mmol Gd³⁺/L (inclusive), particularly in a range of 90 to 600 mmol Gd³⁺/L (inclusive), particularly in a range of 100 to 500 mmol Gd³⁺/L (inclusive), particularly in a range of 150 to 450 mmol Gd³⁺/L (inclusive), more particularly in a range of 200 to 400 mmol Gd³⁺/L (inclusive), and even more particularly in a range of 250 to 350 mmol Gd³⁺/L (inclusive).

In accordance with a sixth embodiment of the first aspect, the present disclosure covers a liquid pharmaceutical formulation, supra, comprising a DO3A-derived tetra-chelate of general formula (I), supra, characterized in that the DO3A-derived tetra-chelate of general formula (I) is a complex between
a DO3A-derived tetra-ligand of general formula (II):

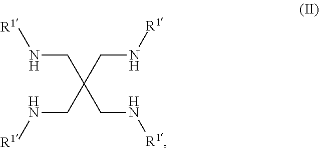

in which
R^{1′} represents a group selected from:

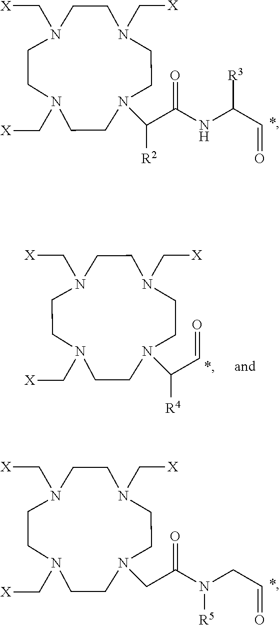

in which group * indicates the point of attachment of said group with the rest of the molecule,
R², R³, and R⁴ independently of each other represents a hydrogen atom or a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
         C₁-C₃-alkyl, C₁-C₃-haloalkyl, and C₁-C₃-alkoxy, and
      wherein said phenyl group is optionally substituted, one, two, or three times, identically or differently, with a halogen atom or a group selected from:
         C₁-C₃-alkyl, C₁-C₃-haloalkyl, and C₁-C₃-alkoxy,
R⁵ represents a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
         C₁-C₃-alkyl, C₁-C₃-haloalkyl, and C₁-C₃-alkoxy, and
      wherein said phenyl group is optionally substituted, one, two or three times, identically or differently, with a halogen atom or a group selected from:
         C₁-C₃-alkyl, C₁-C₃-haloalkyl, and C₁-C₃-alkoxy, and
X represents a group C(═O)OH or C(═O)O⁻,
   or a stereoisomer, a tautomer or a salt thereof, or a mixture of same,
   and ions of a paramagnetic metal M.

In accordance with a seventh embodiment of the first aspect, the present disclosure covers a liquid pharmaceutical formulation, supra, comprising a DO3A-derived tetra-chelate of general formula (I), supra, characterized in that the DO3A-derived tetra-chelate of general formula (I) is a complex between
a DO3A-derived tetra-ligand of general formula (II):

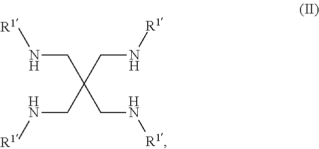

in which
R^{1′} represents a group selected from:

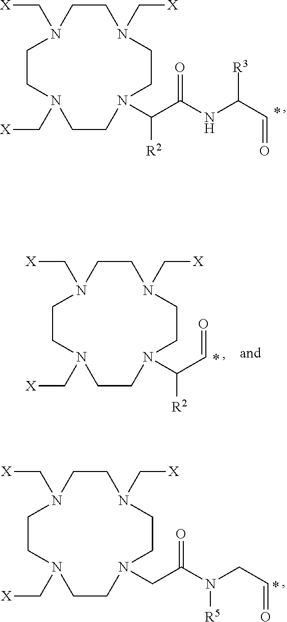

in which group * indicates the point of attachment of said group with the rest of the molecule,
R², R³, and R⁴ independently of each other represents a hydrogen atom or a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
      C₁-C₃-alkyl, C₁-C₃-haloalkyl, and C₁-C₃-alkoxy, and
      wherein said phenyl group is optionally substituted, one, two, or three times, identically or differently, with a halogen atom or a group selected from:
      C₁-C₃-alkyl, C₁-C₃-haloalkyl, and C₁-C₃-alkoxy,
R⁵ represents a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
      C₁-C₃-alkyl, C₁-C₃-haloalkyl, and C₁-C₃-alkoxy, and
      wherein said phenyl group is optionally substituted, one, two or three times, identically or differently, with a halogen atom or a group selected from:
      C₁-C₃-alkyl, C₁-C₃-haloalkyl, and C₁-C₃-alkoxy, and
X represents a group C(═O)OH or C(═O)O⁻,
or a stereoisomer, a tautomer or a salt thereof, or a mixture of same,
and one, two or three ions of a paramagnetic metal M.

In accordance with an eighth embodiment of the first aspect, the present disclosure covers a liquid pharmaceutical formulation, supra, comprising a DO3A-derived tetra-chelate of general formula (I), supra, characterized in that the DO3A-derived tetra-chelate of general formula (I) is a complex between
a DO3A-derived tetra-ligand of general formula (II):

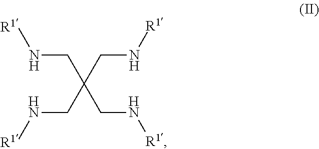

in which
R^{1′} represents a group selected from:

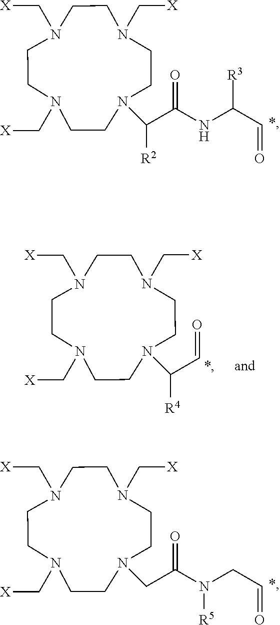

in which group * indicates the point of attachment of said group with the rest of the molecule,
R², R³, and R⁴ independently of each other represents a hydrogen atom or a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
      C₁-C₃-alkyl, C₁-C₃-haloalkyl, and C₁-C₃-alkoxy, and
      wherein said phenyl group is optionally substituted, one, two, or three times, identically or differently, with a halogen atom or a group selected from:
      C₁-C₃-alkyl, C₁-C₃-haloalkyl, and C₁-C₃-alkoxy,
R⁵ represents a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
      C₁-C₃-alkyl, C₁-C₃-haloalkyl, and C₁-C₃-alkoxy, and
      wherein said phenyl group is optionally substituted, one, two or three times, identically or differently, with a halogen atom or a group selected from:
      C₁-C₃-alkyl, C₁-C₃-haloalkyl, and C₁-C₃-alkoxy, and
X represents a group C(═O)OH or C(═O)O—,
or a stereoisomer, a tautomer or a salt thereof, or a mixture of same,
and one, two or three Gd³⁺ ions.

In accordance with a ninth embodiment of the first aspect, the present disclosure covers a liquid pharmaceutical formulation, supra, comprising a DO3A-derived tetra-chelate of formula (I), supra, characterized in that the DO3A-derived tetra-chelate of formula (I) is selected from the complex between
a DO3A-derived tetra-ligand of formula (II-a), (II-b), or (II-c) having structures as follows:

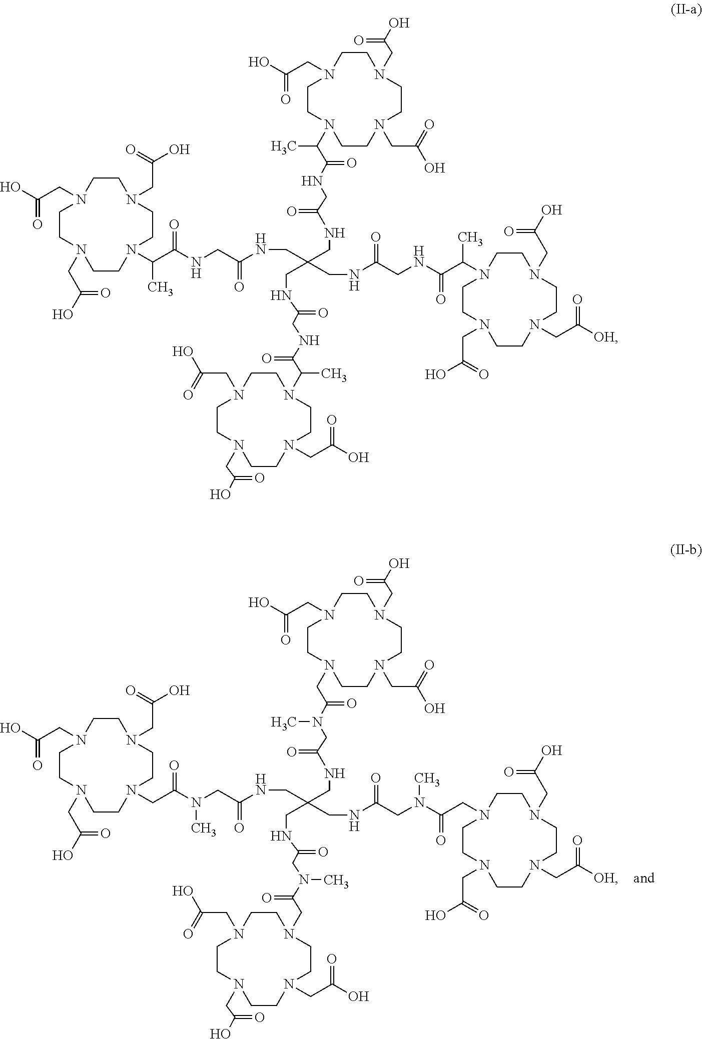 [SMILES: CC(C(=O)NCC(=O)NCC(CNC(=O)CNC(=O)C(C)N1CCN(CC(=O)O)CCN(CC(=O)O)CCN(CC(=O)O)CC1)(CNC(=O)CNC(=O)C(C)N1CCN(CC(=O)O)CCN(CC(=O)O)CCN(CC(=O)O)CC1)CNC(=O)CNC(=O)C(C)N1CCN(CC(=O)O)CCN(CC(=O)O)CCN(CC(=O)O)CC1)N1CCN(CC(=O)O)CCN(CC(=O)O)CCN(CC(=O)O)CC1 ; CN(CC(=O)NCC(CNC(=O)CN(C)C(=O)CN1CCN(CC(=O)O)CCN(CC(=O)O)CCN(CC(=O)O)CC1)(CNC(=O)CN(C)C(=O)CN1CCN(CC(=O)O)CCN(CC(=O)O)CCN(CC(=O)O)CC1)CNC(=O)CN(C)C(=O)CN1CCN(CC(=O)O)CCN(CC(=O)O)CCN(CC(=O)O)CC1)C(=O)CN1CCN(CC(=O)O)CCN(CC(=O)O)CCN(CC(=O)O)CC1 ; "and"]

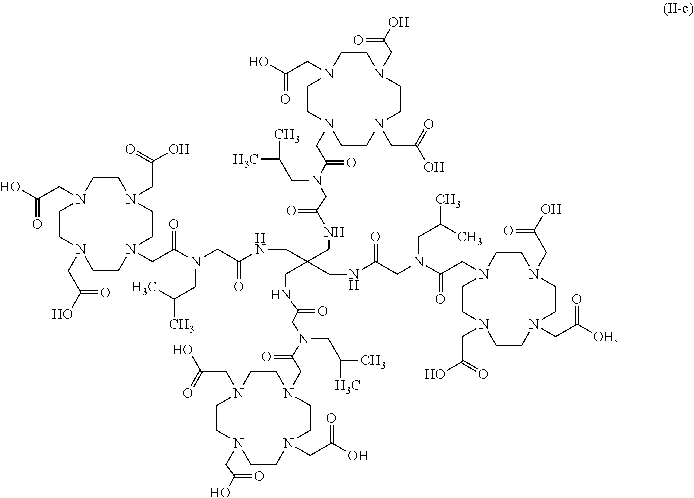 [SMILES: CC(C)CN(CC(=O)NCC(CNC(=O)CN(CC(C)C)C(=O)CN1CCN(CC(=O)O)CCN(CC(=O)O)CCN(CC(=O)O)CC1)(CNC(=O)CN(CC(C)C)C(=O)CN1CCN(CC(=O)O)CCN(CC(=O)O)CCN(CC(=O)O)CC1)CNC(=O)CN(CC(C)C)C(=O)CN1CCN(CC(=O)O)CCN(CC(=O)O)CCN(CC(=O)O)CC1)C(=O)CN1CCN(CC(=O)O)CCN(CC(=O)O)CCN(CC(=O)O)CC1]

or a stereoisomer, a tautomer or a salt thereof, or a mixture of same,
and ions of a paramagnetic metal M.

The paramagnetic metal ion M is chosen from the ions of a paramagnetic metal having an atomic number of 24-29, or 59-70, i.e., chromium (Cr), manganese (Mn), iron (Fe), cobalt (Co), nickel (Ni) or copper (Cu) ions or praseodymium (Pr), neodymium (Nd), promethium (Pm), samarium (Sm), europium (Eu), gadolinium (Gd), terbium (Tb), dysprosium (Dy), holmium (Ho), erbium (Er), thulium (Tm) or ytterbium (Yb) ions. The present listing of metals is intended to include all common oxidation states of the metal ion which exhibit paramagnetism, for example, if the metal is iron, ferrous (Fe²⁺) and ferric (Fe³⁺) ions would be included within the scope. The paramagnetic metal ion M is particularly chosen from manganese, iron, and lanthanide ions, more particularly chosen from the ions Mn²⁺, Fe³⁺, and Gd³⁺ and even more particularly the paramagnetic metal ion M is Gd³⁺.

In accordance with an tenth embodiment of the first aspect, the present disclosure covers a liquid pharmaceutical formulation, supra, comprising a DO3A-derived tetra-chelate of general formula (I), supra, in which the paramagnetic metal ion M is chosen from the ions of a paramagnetic metal having an atomic number of 24-29 or 59-70.

In accordance with a eleventh embodiment of the first aspect, the present disclosure covers a liquid pharmaceutical formulation, supra, comprising a DO3A-derived tetra-chelate of general formula (I), supra, in which the paramagnetic metal ion M is chosen from a lanthanide metal ion.

In accordance with a twelfth embodiment of the first aspect, the present disclosure covers a liquid pharmaceutical formulation, supra, comprising a DO3A-derived tetra-chelate of general formula (I), supra, in which the paramagnetic metal ion M is chosen from the ions Mn²⁺, Fe³⁺, and Gd³⁺ and even more particularly the paramagnetic metal ion M is a Gd³⁺ ion.

The liquid pharmaceutical formulation of the present disclosure may comprise besides the DO3A-derived tetra chelate of general formula (I), supra, the corresponding M₃-DO3A-derived chelate, the corresponding M₂-DO3A-derived chelate, the corresponding M-DO3A-derived chelate and the corresponding DO3A-derived tetra-ligand of general formula (II), and stereoisomers, tautomers, or salts thereof, or mixtures thereof.

The liquid pharmaceutical formulation of the present disclosure may comprise besides the DO3A-derived tetra chelate of general formula (I), supra, one or more of the corresponding M₃-DO3A-derived chelate, the corresponding M₂-DO3A-derived chelate, the corresponding M-DO3A-derived chelate and the corresponding DO3A-derived tetra-ligand of general formula (II), and stereoisomers, tautomers, or salts thereof, or mixtures thereof.

In accordance with an thirteenth embodiment of the first aspect, the present disclosure covers a liquid pharmaceutical formulation, supra, comprising a DO3A-derived tetra-chelate of general formula (I), supra, the corresponding M₃-DO3A-derived chelate, the corresponding M₂-DO3A-derived chelate, the corresponding M-DO3A-derived chelate and the corresponding DO3A-derived tetra-ligand of general formula (II), and stereoisomers, tautomers, or salts thereof, or mixtures thereof.

In accordance with a fourteenth embodiment of the first aspect, the present disclosure covers a liquid pharmaceutical formulation, supra, comprising a Gd₄-DO3A-derived tetra-chelate of general formula (I), supra, the corresponding Gd₃-DO3A-derived chelate, the corresponding Gd₂-DO3A-derived chelate, the corresponding Gd-DO3A-derived chelate and the corresponding DO3A-derived tetra-ligand of general formula (II), and stereoisomers, tautomers, or salts thereof, or mixtures thereof.

In accordance with a fifteenth embodiment of the first aspect, the present disclosure covers a liquid pharmaceutical formulation, supra, comprising a DO3A-derived tetra-chelate of general formula (I), supra, one or more of the corresponding M₃-DO3A-derived chelate, the corresponding M₂-DO3A-derived chelate, the corresponding M-DO3A-derived chelate and the corresponding DO3A-derived tetra-ligand of general formula (II), and stereoisomers, tautomers, or salts thereof, or mixtures thereof.

In accordance with a sixteenth embodiment of the first aspect, the present disclosure covers a liquid pharmaceutical formulation, supra, comprising a Gd₄-DO3A-derived tetra-chelate of general formula (I), supra, one or more of the corresponding Gd₃-DO3A-derived chelate, the corresponding Gd₂-DO3A-derived chelate, the corresponding Gd-DO3A-derived chelate and the corresponding DO3A-derived tetra-ligand of general formula (II), and stereoisomers, tautomers, or salts thereof, or mixtures thereof.

DO3A-derived tetra-chelates with sub-stoichiometric amounts of paramagnetic metal ions are compositions containing DO3A-derived tetra-chelates and further including chelates between a DO3A-derived tetra-ligand of general formula (II) and sub-stoichiometric amounts of paramagnetic metal ions, such as one, two or three paramagnetic metal ions, and/or containing the DO3A-derived tetra-ligand of general formula (II), or mixtures thereof. In various embodiments, the DO3A-derived tetra-ligand comprising sub-stoichiometric amounts of paramagnetic metal ions may include sub-stoichiometric amounts of one or more weak binding metal ions. As used herein, the term “weak binding metal ions” include metal ions for the alkali metals, alkaline earth metals, and transition metals that have a binding affinity for the DO3A-derived tetra-chelate that is less than the binding affinity of the DO3A-derived tetra-chelate and the lanthanide metal ion. In various embodiments, the weak binding metal ions may include lithium, calcium, sodium, zinc, potassium, or magnesium ions. According to various embodiments, the DO3A-derived tetra-chelates with sub-stoichiometric amounts of paramagnetic metal ions may contain 3.95 to 3.9996 mole of paramagnetic metal ion relative to 1.000 mole of DO3A-derived tetra-ligand of general formula (II). This results in a paramagnetic metal scavenging capacity of 0.01 to 1.25% mol/mol (relative to the total concentration of the paramagnetic metal).

Without intending to be bound by any theory, it is believed that sub-stoichiometric chelates, having sub-stoichiometric amounts of paramagnetic metal ions which are contained in the DO3A-derived tetra-chelates, may act as scavengers that will bind to any paramagnetic metal ion that may be decomplexed or released from the DO3A-derived tetra-chelate by binding to the released paramagnetic metal ion in a transmetallation exchange reaction with the weak binding metal ion in the ligating site or by binding the paramagnetic metal ion in the metal-free ligating site, thereby removing the free paramagnetic metal ion from solution. According to various embodiments, the pharmaceutical formulation comprising DO3A-derived tetra-chelates and DO3A-derived tetra-ligands with sub-stoichiometric amounts of paramagnetic metal ions may have concentrations of the DO3A-derived tetra-ligands with sub-stoichiometric amounts of paramagnetic metal ions in the range of 0.01% to 1.25% mol/mol (inclusive), for example in the range of 0.02% to 1% mol/mol (inclusive), more particularly in the range of 0.025% to 0.5% mol/mol (inclusive), this proportion being related to the total concentration of the paramagnetic metal ion in the formulation.

In specific embodiments, the DO3A-derived tetra-chelates with sub-stoichiometric amounts of paramagnetic metal ions may include sub-stoichiometric paramagnetic gadolinium ion (Gd³⁺), such as Gd₄-DO3A-derived tetra-chelates with sub-stoichiometric amounts of paramagnetic gadolinium ions. According to these embodiments, the pharmaceutical formulation comprising Gd₄-DO3A-derived tetra-chelates further comprise chelates between a DO3A-derived tetra-ligand of general formula (II) and one, two or three Gd³⁺ ions, and/or containing the DO3A-derived tetra-ligand of general formula (II) as a free ligand, or mixtures thereof. In various embodiments, the Gd₄-DO3A-derived tetra-ligand comprising sub-stoichiometric amounts of paramagnetic gadolinium ions and/or the free DO3A-derived tetra-ligand may include sub-stoichiometric or stoichiometric amounts, respectively, of one or more weak binding metal ions bound in the gadolinium-free ligating sites.

More specifically a sub-stoichiometric chelate between the DO3A-derived tetra-ligand of formula (II-a),

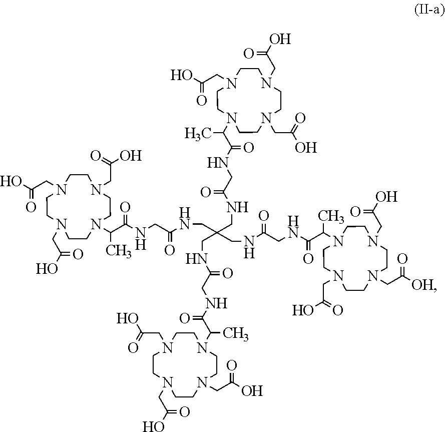 [SMILES: CC(C(=O)NCC(=O)NCC(CNC(=O)CNC(=O)C(C)N1CCN(CC(=O)O)CCN(CC(=O)O)CCN(CC(=O)O)CC1)(CNC(=O)CNC(=O)C(C)N1CCN(CC(=O)O)CCN(CC(=O)O)CCN(CC(=O)O)CC1)CNC(=O)CNC(=O)C(C)N1CCN(CC(=O)O)CCN(CC(=O)O)CCN(CC(=O)O)CC1)N1CCN(CC(=O)O)CCN(CC(=O)O)CCN(CC(=O)O)CC1]

and three Gd³⁺ ions is the Gd₃-DO3A-derived chelate of formula (Gd₃-II-a),

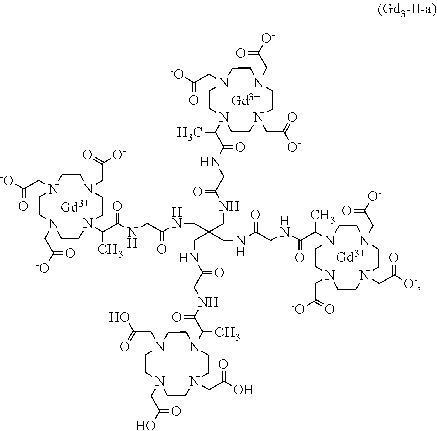

a sub-stoichiometric chelate between the DO3A-derived tetra-ligand of formula (II-a) and two Gd³⁺ ions is the Gd₂-DO3A-derived chelate of formula (Gd₂-II-a),

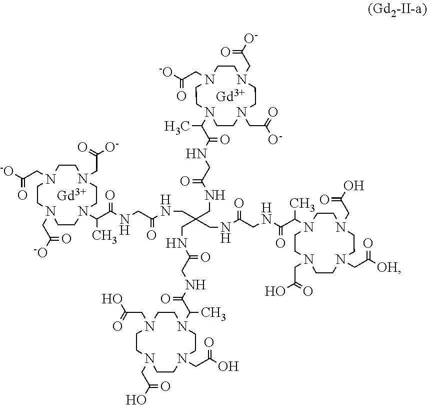

and a sub-stoichiometric chelate between the DO3A-derived tetra-ligand of formula (II-a) and one Gd³⁺ ion is the Gd-DO3A-derived chelate of formula (Gd-II-a),

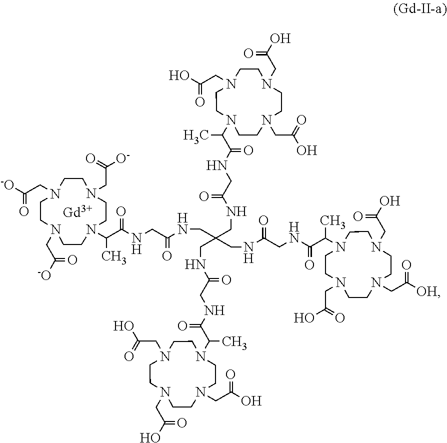

In specific embodiments, the DO3A-derived tetra-chelates with sub-stoichiometric amounts of gadolinium ions (Gd³⁺) are Gd₃-DO3A-derived chelate of formula (Gd₃-II-a), Gd₂-DO3A-derived chelate of formula (Gd₂-II-a), Gd-DO3A-derived chelate of formula (Gd-II-a), or DO3A-derived tetra-ligand of formula (II-a), or mixtures thereof.

More specifically a sub-stoichiometric chelate between the DO3A-derived tetra-ligand of formula (II-b),

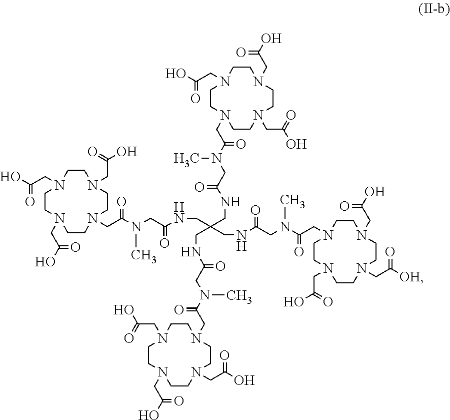 [SMILES: CN(CC(=O)NCC(CNC(=O)CN(C)C(=O)CN1CCN(CC(=O)O)CCN(CC(=O)O)CCN(CC(=O)O)CC1)(CNC(=O)CN(C)C(=O)CN1CCN(CC(=O)O)CCN(CC(=O)O)CCN(CC(=O)O)CC1)CNC(=O)CN(C)C(=O)CN1CCN(CC(=O)O)CCN(CC(=O)O)CCN(CC(=O)O)CC1)C(=O)CN1CCN(CC(=O)O)CCN(CC(=O)O)CCN(CC(=O)O)CC1]

and three Gd³⁺ ions is the Gd₃-DO3A-derived chelate of formula (Gd₃-II-b),

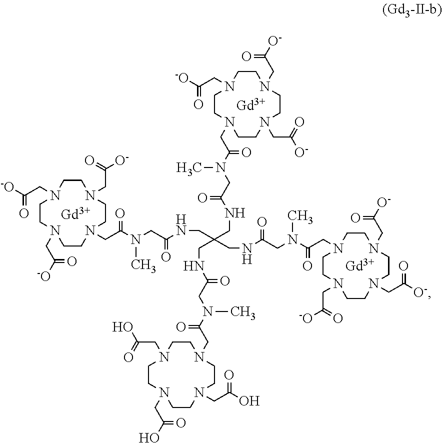

a sub-stoichiometric chelate between the DO3A-derived tetra-ligand of formula (II-b) and two Gd³⁺ ions is the Gd₂-DO3A-derived chelate of formula (Gd₂-II-b),

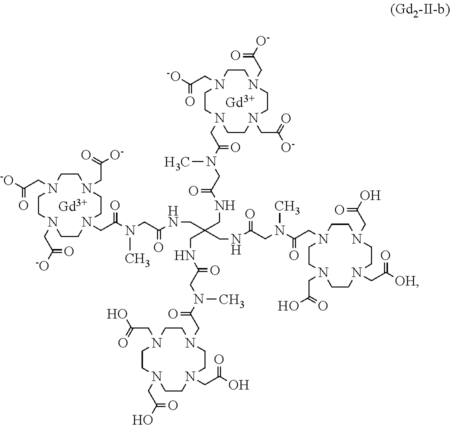

and a sub-stoichiometric chelate between the DO3A-derived tetra-ligand of formula (II-b) and one Gd³⁺ ion is the Gd-DO3A-derived chelate of formula (Gd-II-b),

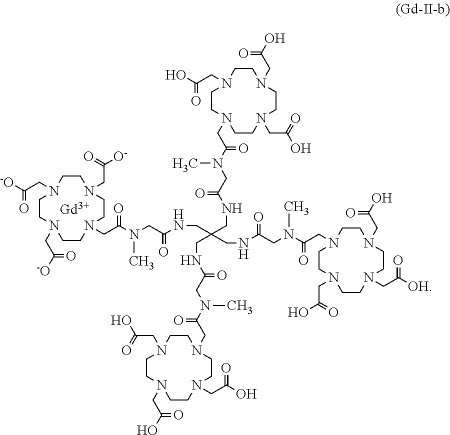

In specific embodiments, the DO3A-derived tetra-chelates with sub-stoichiometric amounts of gadolinium ions (Gd³⁺) are Gd₃-DO3A-derived chelate of formula (Gd₃-II-b), Gd₂-DO3A-derived chelate of formula (Gd₂-II-b), Gd-DO3A-derived chelate of formula (Gd-II-b), or DO3A-derived tetra-ligand of formula (II-b), or mixtures thereof.

More specifically a sub-stoichiometric chelate between the DO3A-derived tetra-ligand of formula (II-c),

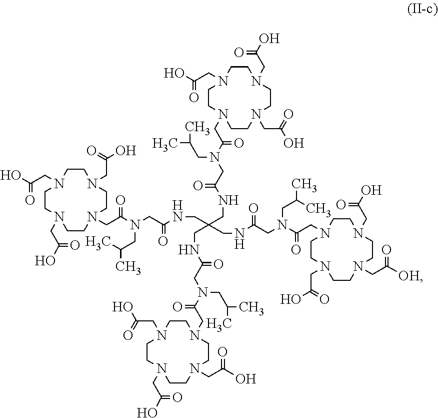 [SMILES: CC(C)CN(CC(=O)NCC(CNC(=O)CN(CC(C)C)C(=O)CN1CCN(CC(=O)O)CCN(CC(=O)O)CCN(CC(=O)O)CC1)(CNC(=O)CN(CC(C)C)C(=O)CN1CCN(CC(=O)O)CCN(CC(=O)O)CCN(CC(=O)O)CC1)CNC(=O)CN(CC(C)C)C(=O)CN1CCN(CC(=O)O)CCN(CC(=O)O)CCN(CC(=O)O)CC1)C(=O)CN1CCN(CC(=O)O)CCN(CC(=O)O)CCN(CC(=O)O)CC1]

and three Gd³⁺ ions is the Gd₃-DO3A-derived chelate of formula (Gd₃-II-c),

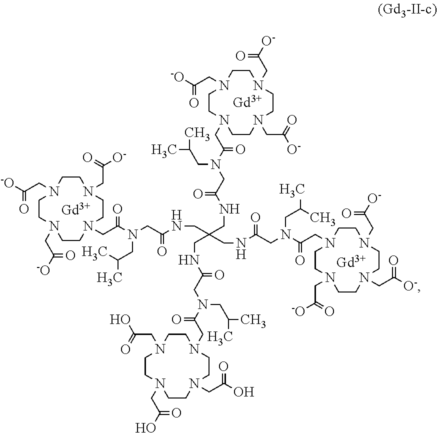

a sub-stoichiometric chelate between the DO3A-derived tetra-ligand of formula (II-c) and two Gd³⁺ ions is the Gd₂-DO3A-derived chelate of formula (Gd₂-II-c),

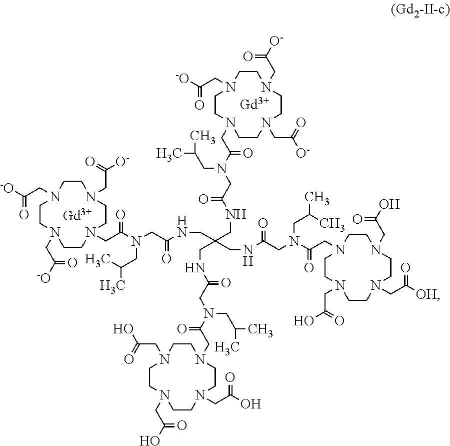

and a sub-stoichiometric chelate between the DO3A-derived tetra-ligand of formula (II-c) and one Gd³⁺ ion is the Gd-DO3A-derived chelate of formula (Gd-II-c),

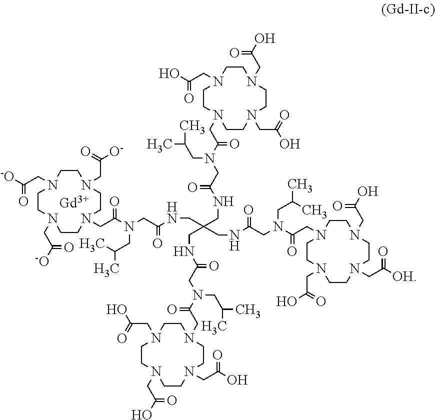

In specific embodiments, the DO3A-derived tetra-chelates with sub-stoichiometric amounts of gadolinium ions (Gd³⁺) are Gd₃-DO3A-derived chelate of formula (Gd₃-II-c), Gd₂-DO3A-derived chelate of formula (Gd₂-II-c), Gd-DO3A-derived chelate of formula (Gd-II-c), or DO3A-derived tetra-ligand of formula (II-c), or mixtures thereof.

The formulation according to the present disclosure exhibits stability over time such that the concentration of free paramagnetic metal ions remains essentially zero (below the detection limit of sensitive analytical methods). In various embodiments, the concentration of free paramagnetic metal ions M remains less than or equal to 2 ppm (m/v), i.e. in the range of 0 to 2 ppm (m/v) (inclusive), over a period of at least 6 months, at 25° C. and 40° C. Accelerated storage condition (6 months at 40° C.) is considered to be an adequate condition for accelerated stress conditions for pharmaceutical contrast formulations.

In accordance with a seventeenth embodiment of the first aspect, the present disclosure covers a liquid pharmaceutical formulation, supra, characterized in that it has a concentration of a free paramagnetic metal ion M of less than or equal to 5 ppm (m/v), i.e. in a range of 0 to 5 ppm (m/v) (inclusive), particularly less than or equal to 2 ppm (m/v), i.e. in a range of 0 to 2 ppm (m/v) (inclusive), and more particularly less than or equal to 0.5 ppm (m/v), i.e. in a range of 0 to 0.5 ppm (m/v) (inclusive).

In accordance with a eighteenth embodiment of the first aspect, the present disclosure covers a liquid pharmaceutical formulation, supra, characterized in that it comprises at least one compound capable of forming a chelate with a free paramagnetic metal ion M contained in the composition of Formula (I). Such compounds, also called “metal scavenging compounds” are described in detail herein. According to various embodiments, the compound capable of forming a chelate with a free paramagnetic metal ion M may have a concentration in the formulation in a range of 0.002% to 5% mol/mol (inclusive), particularly in a range of 0.01% to 1% mol/mol (inclusive), more particularly in a range of 0.05% to 0.5% mol/mol (inclusive), measured as a proportion relative to the total paramagnetic metal ion, such as Gd³⁺ concentration in the formulation.

In accordance with a nineteenth embodiment of the first aspect, the present disclosure covers a liquid pharmaceutical formulation, supra, characterized in that it comprises one or more compounds capable of forming a complex with the free paramagnetic metal ion M, which compounds may be selected from Ca-BT-DO3A (Calcobutrol), Ca-DOTA, Ca-HP-DO3A and Ca-DTPA or from the respective free chelating ligands, or salts thereof with alkaline metals, alkaline earth metals, weak binding transition metals, or organic bases.

In accordance with a twentieth embodiment of the first aspect, the present disclosure covers a liquid pharmaceutical formulation, supra, characterized in that it comprises a compound capable of forming a complex with the free paramagnetic metal ion M, which compound is Ca-BT-DO3A (Calcobutrol).

In accordance with a twenty-first embodiment of the first aspect, the present disclosure covers a liquid pharmaceutical formulation, supra, characterized in that it comprises a compound capable of forming a chelate with the free paramagnetic metal ion M, which compound is Ca-BT-DO3A (Calcobutrol), preferably in the range of 0.002% to 5% mol/mol (inclusive), measured as a proportion relative to a total paramagnetic metal ion concentration, such as Gd³⁺ concentration in the formulation.

In accordance with an twenty-second embodiment of the first aspect, the proportion of Ca-BT-DO3A is in a range of 0.002% to 5% mol/mol (inclusive), for example in a range of 0.002% to 1% mol/mol (inclusive), particularly in a range of 0.01% to 1% mol/mol (inclusive), more particularly in a range of 0.05% to 0.5% mol/mol (inclusive), this proportion being related to the total paramagnetic metal ion concentration, such as Gd³⁺, in said formulation.

In accordance with a twenty-third embodiment of the first aspect, the present disclosure covers a liquid pharmaceutical formulation, supra, characterized in that the formulation comprises compounds capable of forming complexes with the free paramagnetic metal ion M, which compounds are DO3A-derived tetra-chelates with sub-stoichiometric amounts of paramagnetic metal ions, as defined supra, or salts thereof with Ca²⁺ ions, Na⁺ ions, Zn²⁺ ions, Mg²⁺ ions, and/or meglumine ions.

In accordance with a twenty-fourth embodiment of the first aspect, the present disclosure covers a liquid pharmaceutical formulation, supra, characterized in that the formulation comprises compounds capable of forming complexes with the free paramagnetic metal ion M, which compounds are DO3A-derived tetra-chelates with sub-stoichiometric amounts of Gd³⁺ ions, as defined supra, or salts thereof with Ca²⁺ ions, Na⁺ ions, Zn²⁺ ions, Mg²⁺ ions and/or meglumine ions.

In accordance with a twenty-fifth embodiment of the first aspect, the proportion of DO3A-derived tetra-chelates with sub-stoichiometric amounts of Gd³⁺ ions is in a range of 0.002% to 5% mol/mol (inclusive) [with reference to the total molar Gd-concentration].

In accordance with a twenty-sixth embodiment of the first aspect, the present disclosure covers a liquid pharmaceutical formulation, supra, characterized in that it comprises compounds capable of forming a chelate with the free paramagnetic metal ion M, which compounds are DO3A-derived tetra-chelates with sub-stoichiometric amounts of Gd³⁺ ions, which are selected from:

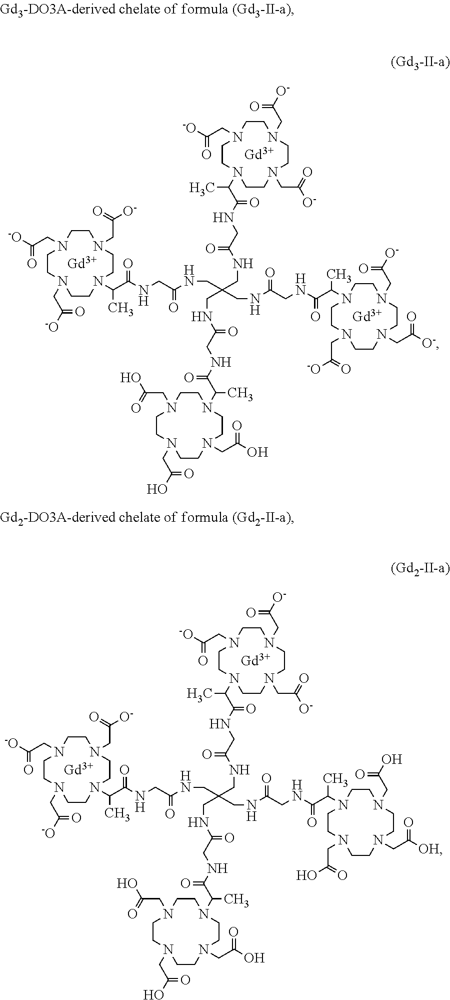

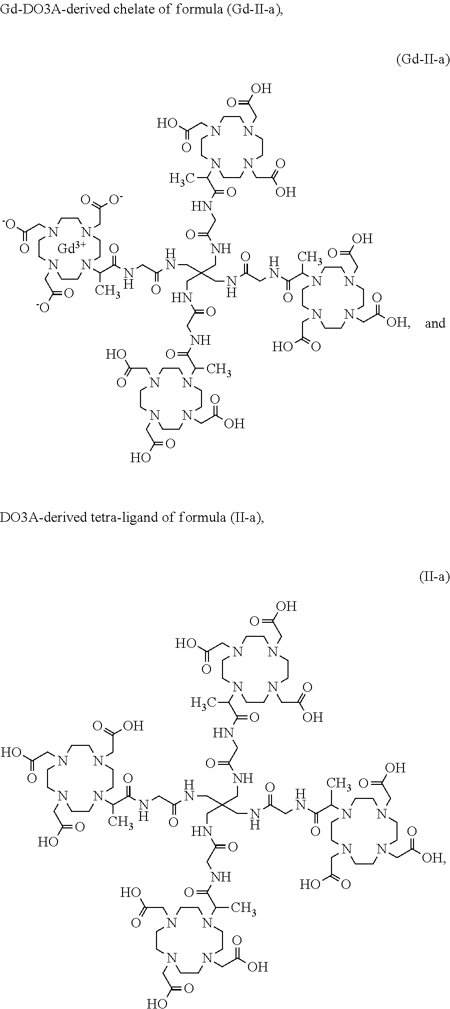

or stereoisomers, tautomers or salts thereof, or mixtures of same,
preferably in the range of of 0.01 to 1.25 mol % [with reference to the total molar Gd-concentration].

In accordance with a twenty-seventh embodiment of the first aspect, the present disclosure covers a liquid pharmaceutical formulation, supra, characterized in that it comprises compounds capable of forming a chelate with the free paramagnetic metal ion M, which compounds are DO3A-derived tetra-chelates with sub-stoichiometric amounts of Gd³⁺ ions, which are selected from

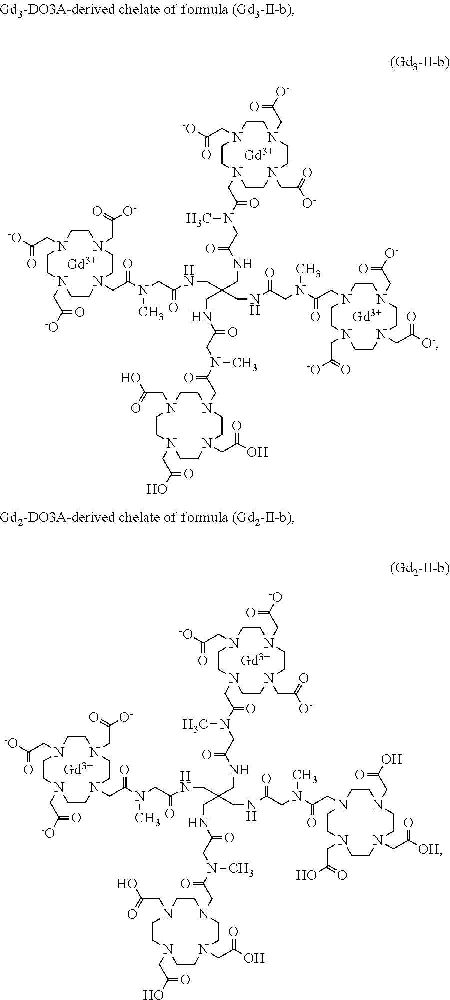

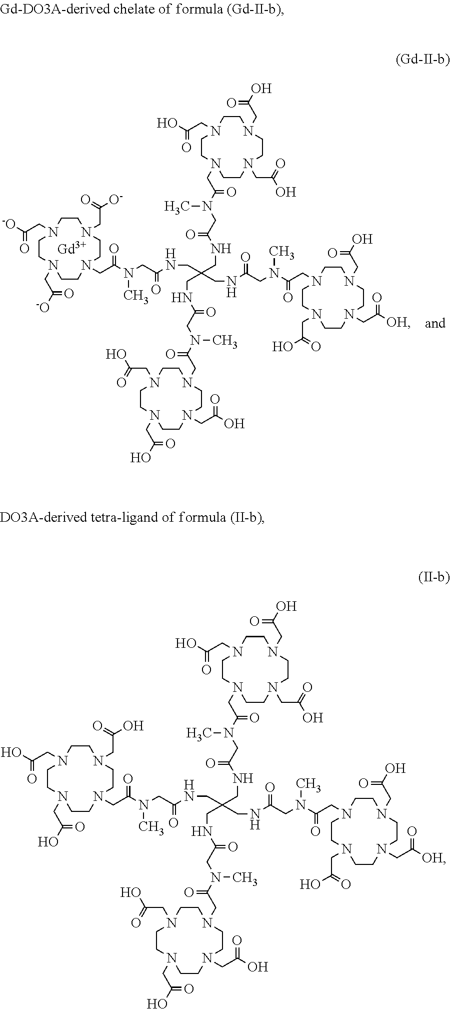

or stereoisomers, tautomers or salts thereof, or mixtures of same,
preferably in the range of of 0.01 to 1.25 mol % [with reference to the total molar Gd-concentration].

In accordance with a twenty-eighth embodiment of the first aspect, the present disclosure covers a liquid pharmaceutical formulation, supra, characterized in that it comprises compounds capable of forming a chelate with the free paramagnetic metal ion M, which compounds are DO3A-derived tetra-chelates with sub-stoichiometric amounts of Gd³⁺ ions, which are selected from

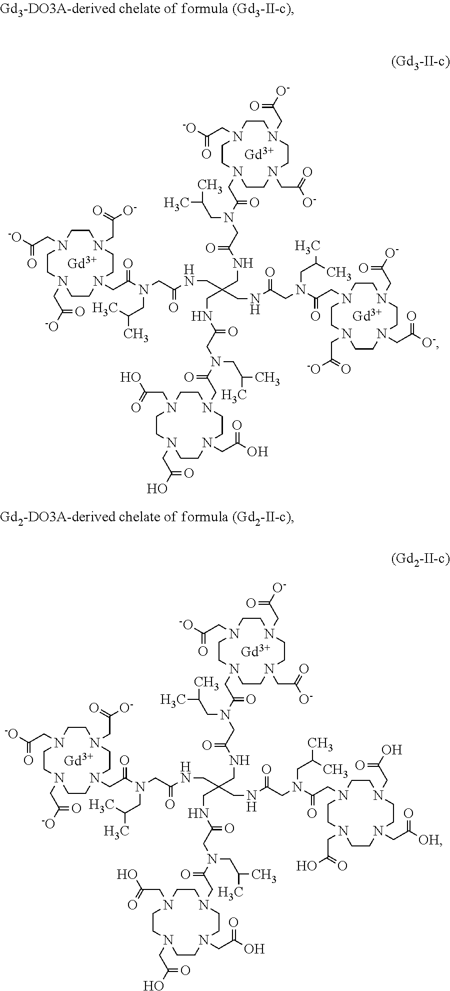

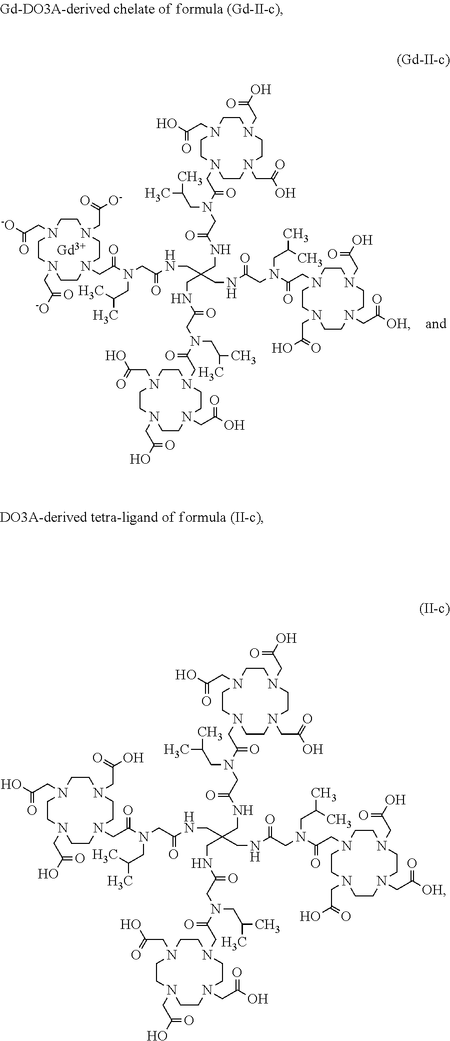

or stereoisomers, tautomers or salts thereof, or mixtures of same,
preferably in the range of of 0.01 to 1.25 mol % [with reference to the total molar Gd-concentration].

In accordance with a twenty-ninth embodiment of the first aspect, the present disclosure covers a liquid pharmaceutical formulation comprising a DO3A-derived tetra-chelate of general formula (I):

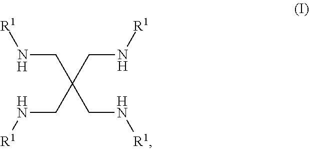

in which
R¹ represents a group selected from:

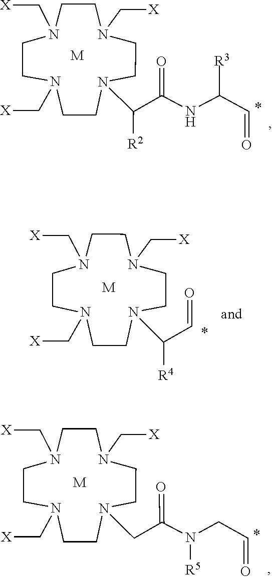

in which group * indicates the point of attachment of said group with the rest of the molecule,
R², R³, and R⁴ independently of each other represents a hydrogen atom or a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
         C₁-C₃-alkyl, C₁-C₃-haloalkyl, and C₁-C₃-alkoxy, and
      wherein said phenyl group is optionally substituted, one, two, or three times, identically or differently, with a halogen atom or a group selected from:
         C₁-C₃-alkyl, C₁-C₃-haloalkyl, and C₁-C₃-alkoxy,
R⁵ represents a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
         C₁-C₃-alkyl, C₁-C₃-haloalkyl, and C₁-C₃-alkoxy, and
      wherein said phenyl group is optionally substituted, one, two, or three times, identically or differently, with a halogen atom or a group selected from:
         C₁-C₃-alkyl, C₁-C₃-haloalkyl, and C₁-C₃-alkoxy,
X represents a group C(═O)O—, and
M represents an ion of a paramagnetic metal,
or a stereoisomer, a tautomer, a salt thereof, or a mixture of same,
said formulation further comprising
   a sub-stoichiometric chelate, as described herein, in a concentration fraction of 0.01 to 1.25 mol % [with reference to the total molar concentration of the paramagnetic metal], wherein the sub-stoichiometric chelate is selected from one or more of M₃-DO3A-derived chelate, M₂-DO3A-derived chelate, M-DO3A-derived chelate, and the DO3A-derived tetra-ligand, or mixtures thereof or stereoisomers, tautomers, salts thereof, or a mixture of any thereof,
      wherein the one, two or three DO3A ligands of said sub-stoichiometric chelates, which are not complexed with ions of paramagnetic metals, and the four DO3A ligands of the DO3A-derived tetra-ligand, may be present as complexes or salts with Ca²⁺ ions, Na⁺ ions, Zn²⁺ ions, Mg²⁺ ions, or meglumine ions or as free carboxylic acids,
   a pharmaceutically acceptable solvent, and
   optionally comprising a buffer,
wherein the DO3A-derived tetra-chelate of general formula (I) has a concentration in the formulation in a range of 1 mmol paramagnetic metal ion/L to 1000 mmol paramagnetic metal ion/L (inclusive), particularly in a range of 60 to 750 mmol paramagnetic metal ion/L (inclusive), particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive).

In accordance with a thirties embodiment of the first aspect, the present disclosure covers a liquid pharmaceutical formulation comprising a DO3A-derived tetra-chelate of general formula (I):

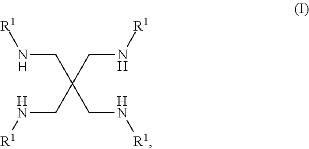

in which
R¹ represents a group selected from:

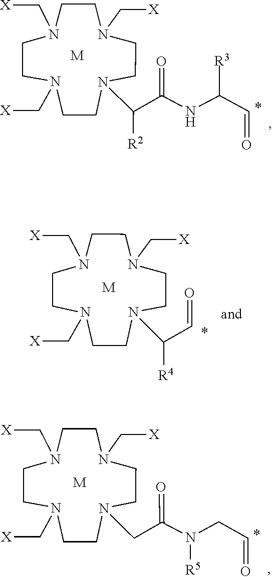

in which group * indicates the point of attachment of said group with the rest of the molecule,
R², R³, and R⁴ independently of each other represents a hydrogen atom or a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
         C₁-C₃-alkyl, C₁-C₃-haloalkyl, and C₁-C₃-alkoxy, and wherein said phenyl group is optionally substituted, one, two, or three times, identically or differently, with a halogen atom or a group selected from:
         C₁-C₃-alkyl, C₁-C₃-haloalkyl, and C₁-C₃-alkoxy,
R⁵ represents a group selected from:
   C₁-C₆-alkyl, C₃-C₆-cycloalkyl, (C₁-C₂-alkoxy)-(C₂-C₃-alkyl)-, and phenyl,
      wherein said C₁-C₆-alkyl group is optionally substituted, identically or differently, with a phenyl substituent, which phenyl substituent is optionally substituted, one, two, or three times, identically or differently, with a halogen atom or a group selected from:
         C₁-C₃-alkyl, C₁-C₃-haloalkyl, and C₁-C₃-alkoxy, and
      wherein said phenyl group is optionally substituted, one, two, or three times, identically or differently, with a halogen atom or a group selected from:
         C₁-C₃-alkyl, C₁-C₃-haloalkyl, and C₁-C₃-alkoxy,
X represents a group C(═O)O—, and
each M represents a Gd³⁺ ion,
or a stereoisomer, a tautomer, a salt thereof, or a mixture of same,
said formulation further comprising
   a sub-stoichiometric chelate, as described herein, in a concentration fraction of 0.01 to 1.25 mol % [with reference to the total molar Gd-concentration], wherein the sub-stoichiometric chelate is selected from one or more of Gd₃-DO3A-derived chelate, Gd₂-DO3A-derived chelate, Gd-DO3A-derived chelate, and the DO3A-derived tetra-ligand, or mixtures thereof or stereoisomers, tautomers, salts thereof, or a mixture of any thereof,
      wherein the one, two or three DO3A ligands of said sub-stoichiometric chelates, which are not complexed with Gd³⁺ ions, and the four DO3A ligands of the DO3A-derived tetra-ligand, may be present as complexes or salts with Ca²⁺ ions, Na⁺ ions, Zn²⁺ ions, Mg²⁺ ions, or meglumine ions or as free carboxylic acids,
   a pharmaceutically acceptable solvent, and
   optionally comprising a buffer,
wherein the DO3A-derived tetra-chelate of general formula (I) has a concentration in the formulation in a range of 1 mmol Gd³⁺/L to 1000 mmol Gd³⁺/L (inclusive), particularly in a range of 60 to 750 mmol Gd³⁺/L (inclusive), particularly in a range of 70 to 700 mmol Gd³⁺/L (inclusive), particularly in a range of 80 to 650 mmol Gd³⁺/L (inclusive), particularly in a range of 90 to 600 mmol Gd³⁺/L (inclusive), particularly in a range of 100 to 500 mmol Gd³⁺/L (inclusive), particularly in a range of 150 to 450 mmol Gd³⁺/L (inclusive), more particularly in a range of 200 to 400 mmol Gd³⁺/L (inclusive), and even more particularly in a range of 250 to 350 mmol Gd³⁺/L (inclusive).

Owing in particular to the binding of free paramagnetic metal by BT-DO3A (Butrol) or any other free ligand such as DOTA (1,4,7,10-tetraazacyclododecane tetraacetic acid), DTPA (diethylenetriamine pentaacetic acid), and HP-DO3A (2-hydroxypropyl-1,4,7,10-tetraazacyclododecane triacetic acid), according to certain embodiments, the formulation which is the subject of one or more of the various embodiments of the present disclosure may also comprise a complex between BT-DO3A or any other free ligand and a weak binding metal, optionally including meglumine or other cationic agent, particularly in the range of 0.002% to 0.5% mol/mol (inclusive), particularly in the range of 0.01% to 0.5% mol/mol (inclusive), this proportion being related to the total paramagnetic metal ion concentration, such as Gd³⁺ concentration, in said formulation. Particularly, the complex between BT-DO3A or any other free ligand and a paramagnetic metal ion is a paramagnetic metal chelate. The nature of the metal chelated by BT-DO3A or any other free ligand is predominantly the same as that of the paramagnetic metal chelated by the chelating ligand of the complex of formula (I). However, the formulation according to various embodiments of the disclosure may also comprise a small proportion of free BT-DO3A and/or a complex between BT-DO3A and a metal other than the one chelated by the chelating ligand of the complex of formula (I). The formulation can thus also comprise a complex between BT-DO3A and an ion of any metal that can be extracted from the containers, for example from a surface of a glass, plastic, or metal reaction or storage container, in which the formulation is prepared and/or stored, for example iron, copper, and/or magnesium ion.

Liquid pharmaceutical formulations comprising Gd₄-DO3A-derived tetra chelates of general formula (I), as defined supra, display a high relaxivity, which is a measure for the efficiency in MRI imaging procedures, and an improved mass efficiency (cost of industrial production). According to various embodiments, the formulations may display relaxivity values for r₁ in the range of 10 to 14 L mmol⁻¹ s⁻¹ Gd⁻¹ (at 1.41 T, human plasma). The observed relaxivities may range from 2-3 fold higher than those associated with formulations comprising conventional MRI contrast agents, in particular gadobutrol and gadopentetate dimeglumine. The DO3A-derived tetra chelates of general formula (I) are highly suitable for high magnetic field imaging (for example for fields of 3 Tesla). The high relaxivities observed with the pharmaceutical formulations comprising the Gd₄-DO3A-derived tetra chelates may allow for improved image quality at lower dosages of paramagnetic metal concentrations. Further, in certain embodiments, lower dosing for the contrast agent may allow for formulations having reduced molar concentrations of the contrast agent in the pharmaceutically acceptable solvent. According to these embodiments, formulations with reduced molar concentrations of the contrast agent may display reduced viscosities compared to conventional MRI contrast agent formulations having higher molar concentrations of contrast agent. Formulations with reduced viscosities may allow for easier administration of equivalent amounts of paramagnetic metal ion with better mixing characteristics with co-administered saline solutions and without significant fluid flow fluctuations, for example when transitioning from injection of high viscosity contrast agent formulation to lower viscosity saline solutions.

The Gd₄-DO3A-derived tetra chelates of general formula (I) exhibit several functional characteristics which are particularly outstanding by themselves or in any combination:

In particular, said DO3A-derived tetra chelates according to the various embodiments of the present disclosure have been found to exhibit one or more of:
a high relaxivity
a favorable pharmacokinetic profile
a fast and complete excretion
a high stability
a high solubility
the potential for a significant dose reduction and the potential for whole body imaging.

The compounds described in EP 1931673B1 and Fries P. et al., Invest. Radiol., 2015 December; 50(12):835-42 are having an average hydration number of above 1 (q>1). The increase of the number of inner sphere water molecules is known to increase the relaxivities but it is also known to decrease the stability of the Gd-chelates (Caravan P., Chem. Soc. Rev., 2006, 35, 512-523; Raymond et al, Bioconjugate Chem., 2005, 16, 3-8).

The compounds according to various embodiments of the present disclosure have only one water molecule directly coordinated to the gadolinium within the complex and have a very high stability (q=1).

The viscosity of the formulations of various embodiments of the current disclosure has been found to be only slightly higher than for sodium chloride solution. In certain embodiments of the present disclosure the osmolality of the formulation may be similar to isotonic sodium chloride solution or blood plasma (275 to 295 mOsm/kg, Pediatr. Nephrol. 2018 Sep. 13); i.e. in the range of 200 to 400 mOsm/kg (inclusive), particularly in the range of 250 to 350 mOsm/kg (inclusive), and is considered low compared to other conventional MRI contrast agents. Isotonic formulations for intravenous administration of contrast agents may be of advantage by not having a significant effect on the distribution of water between intracellular and extracellular spaces compared to non-isotonic solutions, such as hypo- or hypertonic solutions. A comparison of the viscosity and osmolality of embodiments of the present formulations and other conventional MRI contrast agents is presented in Table 1. The combination of low viscosity and the isotonicity with blood of the various embodiments of the formulation of the current disclosure leads to a good local tolerance of the intravenous bolus application and allows a convenient and reproducible application through a long and thin catheter during hand injection (less pressure required) and more consistent flow profiles, for example during fluid flow transitions between contrast agent and saline.

TABLE 1
Viscosities and osmolalities of a formulation containing
the Gd₄-DO3A-derived tetra-chelate of formula
(I-a) and of marketed contrast media:

| | Viscosity | Viscosity | |
|---|---|---|---|
| | 20° C. | 37° C. | Osmolality |
| Product | [mPas] | [mPas] | [mOsm/kg] |
| 0.9% (m/v) sodium chloride | 1.03* | 0.74* | 287** |
| Formulation containing | 1.87* | 1.22* | 294* |
| Gd₄-DO3A-derived tetra- | | | |
| chelate of formula (I-a) | | | |
| Gadovist ® 1.0 | 8.86*** | 4.96*** | 1603*** |
| Dotarem ® | 3.4^{#} | 2.4^{#} | 1350^{#} |
| ProHance ® | 2.0^{##} | 1.3^{##} | 630^{##} |
| Magnevist ® | 4.9^{##} | 2.9^{##} | 1960^{##} |
| Omniscan ® | 2.0^{##} | 1.4^{##} | 789^{##} |
| OptiMARK ® | 3.1^{##} | 2.0^{##} | 1110^{##} |
| MultiHance ® | 9.2^{##} | 5.3^{##} | 1970^{##} |

*Experimental Section, Example 3
**Pediatr. Nephrol. 2018 Sep. 13
***Fachinformation des Arzneimittel-Kompendium der Schweiz ®
^{#}Highlights of Prescribing Information (https://www.accessdata.fda.gov/drugsatfda_docs/label/2017/204781s001lbl.pdf)
^{##}J. Magn. Res. Imaging, 25, 884-899, 2007

In accordance with a thirty-first embodiment of the first aspect, the pH of the formulations may be in a range of 4.5 to 8.5 (inclusive), particularly in a range of 6.6 to 8.0 (inclusive), more particularly in a range of 6.9 to 7.9 (inclusive), more particularly in a range of 7.2 to 7.6. more particularly wherein the pH is 7.4. Formulations having a pH within these ranges make it possible in particular to have an isohydric solution in comparison to in vivo conditions (pH 7.4).

In accordance with a thirty-second embodiment of the first aspect, the formulations according to various embodiments of the present disclosure may be buffered, i.e. it comprises at least one buffer chosen from buffers established for the pH range of 4.5 to 8.5 (inclusive), which buffers are chosen from citrate, lactate, acetate, tartrate, malate, maleate, phosphate, succinate, ascorbate, carbonate, trometamol (TRIS, 2-amino-2-(hydroxymethyl)propane-1,3-diol), HEPES (2-[4-(2-hydroxyethyl)-1-piperazine]ethanesulfonic acid) and MES (2-morpholinoethanesulfonic acid) and mixtures thereof, and particularly the buffer is trometamol.

### Process for Preparing a Pharmaceutical Formulation of the Contrast Agent

In accordance with a second aspect, the present disclosure covers a process for preparing a liquid pharmaceutical formulation according to the present disclosure, which process comprises the following steps:
a) Providing a pharmaceutically acceptable solvent;
b) Optionally dissolving a buffer, thereby obtaining a buffered solution and optionally adjusting the pH of the solution to a pH in a range of 7.6 to 8.2 (inclusive);
c) Optionally dissolving a compound capable of forming a chelate with any free paramagnetic metal ion M;
d) Dissolving a DO3A-derived tetra-chelate of formula (I) as defined supra, in sufficient amount to produce a final solution having a formulation having a concentration of DO3A-derived tetra-chelate of formula (I) in a range of 1 to 1000 mmol paramagnetic metal ion/L (inclusive), wherein the ion of the paramagnetic metal is not Gd³⁺; and in sufficient amount to produce a final solution having a formulation having a concentration of the DO3A-derived tetra-chelate of formula (I) in a range of 60 to 750 mmol paramagnetic metal ion/L (inclusive), wherein the ion of the paramagnetic metal can also be Gd³⁺, particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive).
e) Optionally dissolving an isotonicity agent in the solution;
f) Optionally adjusting the pH of the solution to a pH in a range of 4.5 to 8.5 (inclusive);
g) Optionally adjusting the concentration of said chelate of said formula (I) by addition of an additional amount of the pharmaceutically acceptable solvent; and
h) Optionally sterilizing the solution.

In accordance with a second embodiment of the second aspect, the present disclosure covers a process for preparing a formulation according to various embodiments of the present disclosure, which process comprises the following steps:
a) Providing a pharmaceutically acceptable solvent;
b) Optionally dissolving a buffer, thereby obtaining a buffered solution and optionally adjusting the pH of the solution to a pH in a range of 7.6 to 8.2 (inclusive);
c) Dissolving a compound capable of forming a chelate with any free paramagnetic metal ion M;
d) Dissolving a DO3A-derived tetra-chelate of formula (I) as defined supra, in sufficient amount to produce a final solution having a formulation having a concentration of the DO3A-derived tetra-chelate of formula (I) in a range of 1 to 1000 mmol paramagnetic metal ion/L (inclusive), particularly in a range of 60 to 750 mmol paramagnetic metal ion/L (inclusive), particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive);
e) Optionally dissolving an isotonicity agent in the solution;
f) Optionally adjusting the pH of the solution to a pH in a range of 4.5 to 8.5 (inclusive);
g) Optionally adjusting the concentration of said chelate of said formula (I) by addition of an additional amount of the pharmaceutically acceptable solvent; and
h) Optionally sterilizing the solution.

According to the various embodiments, the order of steps b), c), d) and e) is interchangeable. That is, when performing the method, the lettered identifiers (i.e., a), b), c), etc.) are not intended to indicate a specific order of conducting the steps of the method.

According to various embodiments, the order of steps f) and g) is interchangeable.

The term “pharmaceutically acceptable solvent” is intended to include solvents which are suitable with parenteral application, i.e. with intravenous injection. Particularly, this solvent may be water for injection, or a saline solution, more particularly water for injection.

The term “buffer solution” is intended to mean a solution in a pharmaceutically acceptable solvent which comprises a buffer established for a pH range 4.5 to 8.5 (inclusive), particularly a range of 6.6 to 8.0 (inclusive), more particularly a range of 6.9 to 7.9 (inclusive), more particularly a range of 7.2 to 7.6. Even more particularly the pH is adjusted to a value of 7.4. The buffer used in step b) is chosen from citrate, lactate, acetate, tartrate, malate, maleate, phosphate, succinate, ascorbate, carbonate, trometamol (TRIS, 2-amino-2-(hydroxy-methyl)propane-1,3-diol), HEPES (2-[4-(2-hydroxyethyl)-1-piperazine]ethanesulfonic acid), and MES (2-morpholinoethanesulfonic acid) and mixtures thereof, and particularly the buffer is trometamol.

The adjustment of the pH, for example in step b), may be carried out by adding one of the buffers mentioned above and/or raising the pH by adding an aqueous solution of a base (e.g., sodium hydroxide or meglumine) or lowering the pH by adding an aqueous solution of an acid (e.g., hydrochloric acid).

The compound capable of forming a chelate with any free paramagnetic metal ion M added in step c) is chosen from Ca-BT-DO3A (Calcobutrol), Ca-DOTA, Ca-HP-DO3A and Ca-DTPA or from the respective free ligands, or salts thereof with alkaline metals, alkaline earth metals, weakly bound transition metals or organic bases. Particularly the compound capable of forming a complex with any free paramagnetic metal ion M is Ca-BT-DO3A (Calcobutrol), preferably in a range of 0.002% to 5% mol/mol (inclusive), measured as a proportion relative to the total Gd concentration in the formulation.

The step c) of addition of Ca-BT-DO3A is advantageously carried out at a temperature range of 15 to 60° C. (inclusive), preferably 15 to 40° C. (inclusive).

The DO3A-derived tetra-chelate of formula (I) added in step d) is preferably a Gd₄-DO3A-derived tetra-chelate, particularly selected from the chelates of formulae (I-a), (I-b) and (I-c), as defined supra, more particularly the DO3A-derived tetra-chelate of formula (I) is the Gd₄-DO3A-derived tetra-chelate of formula (I-a), as defined supra.

The DO3A-derived tetra-chelate of formula (I) is added in step d) at a temperature range of 15 to 60° C. (inclusive), particularly 15 to 40° C. (inclusive), in sufficient amount to produce a final solution having a liquid pharmaceutical formulation having a concentration of DO3A-derived tetra-chelate of formula (I) in a range of 1 to 1000 mmol paramagnetic metal ion/L (inclusive) wherein the paramagnetic metal ion is not Gd³⁺, or when a compound capable of forming a complex with any free paramagnetic metal ion M is added in step c) the paramagnetic metal ion can be Gd³⁺; and in sufficient amount to produce a final solution having a liquid pharmaceutical formulation having a concentration of DO3A-derived tetra-chelate of formula (I) in a range of 60 to 750 mmol paramagnetic metal ion/L (inclusive), wherein the paramagnetic metal ion can also be Gd³⁺, particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive).

The fact, that the mixing step d) can be performed without heating is of advantage, because chemical reactions/decomposition which would result in potentially toxic products are avoided.

The isotonicity agent added in step e) particularly is sodium chloride.

The amount of sodium chloride added in step e) is added in order to produce a formulation, which preferably is isotonic to blood plasma.

The pH of the solution in step f) is adjusted to a pH in a range of pH 4.5 to 8.5 (inclusive), particularly in a range of 6.6 to 8.0 (inclusive), more particularly in a range of 6.9 to 7.9 (inclusive), more particularly in a range of 7.2 to 7.6. Even more particularly the pH is adjusted to a value of 7.4.

Step f) of adjustment of the pH is particularly carried out by adding one of the buffers mentioned above and/or by adding an aqueous solution of a base (e.g. sodium hydroxide or meglumine) or an aqueous solution of an acid (e.g. hydrochloric acid).

Step g) of adjustment of the concentration of said chelate of said formula (I), is particularly carried out after measurement of the density of the formulation, by addition of a pharmaceutically acceptable solvent. The target concentration of chelate of formula (I) in the formulation is in a range of 1 to 1000 mmol paramagnetic metal ion/L (inclusive) wherein the ion of the paramagnetic metal is not Gd³⁺, or when a compound capable of forming a complex with any free paramagnetic metal ion M is added in step c) the paramagnetic metal ion can be Gd³⁺; and in sufficient amount to produce a final solution having a liquid pharmaceutical formulation having a concentration of DO3A-derived tetra-chelate of formula (I) in a range of 60 to 750 mmol paramagnetic metal ion/L (inclusive), wherein the ion of the paramagnetic metal can also be Gd³⁺, particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive).

The step g) of adjustment of the concentration of the chelate of formula (I) as defined supra is particularly a step of adjustment of the volume by adding pharmaceutically acceptable solvent so as to adjust the density of the liquid formulation to a density in a range of 1.0 to 1.3 g·cm⁻³ (inclusive), particularly in a range of 1.0 to 1.2 g·cm⁻³, more particularly to a density in a range of 1.075 to 1.125 g·cm⁻³.

Step h) of sterilization of the formulation is carried out according to methods known to those skilled in the art.

In accordance with a third embodiment of the second aspect, the present disclosure covers a process for preparing a liquid pharmaceutical formulation, wherein the step of dissolving the DO3A-derived tetra-chelate of formula (I) comprises:
dissolving a DO3A-derived tetra-chelate of formula (I) as defined supra, in a pharmaceutically acceptable solvent or an aqueous buffer to provide a first solution, wherein the DO3A-derived tetra-chelate of formula (I) is dissolved in sufficient amount to produce a liquid pharmaceutical formulation having a concentration of the DO3A-derived tetra-chelate of formula (I) in a range of 1 to 1000 mmol paramagnetic metal ion/L (inclusive), wherein the ion of the paramagnetic metal is not Gd³⁺; and in a range of 60 to 750 mmol paramagnetic metal ion/L (inclusive), wherein the ion of the paramagnetic metal can also be Gd³⁺, particularly in a range of 70 to 700 mmol paramagnetic metal ion/L (inclusive), particularly in a range of 80 to 650 mmol paramagnetic metal ion/L (inclusive), particularly in a range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive).

In accordance with a fourth embodiment of the second aspect, the present disclosure covers a process for preparing a formulation according to the present disclosure, wherein the process comprises:
dissolving an amount of the compound capable of forming a chelate with free paramagnetic metal ion M in the range of 0.002% to 5% mol/mol (inclusive) with reference to the total concentration of the paramagnetic metal ion in the formulation in the pharmaceutically acceptable solvent or an aqueous buffer solution to provide a first solution;
dissolving the DO3A-derived tetra-chelate of formula (I) as defined supra, in the first solution in sufficient amount to produce a final solution having a liquid pharmaceutical formulation having a concentration of the DO3A-derived tetra-chelate of formula (I) in a range of 1 mmol paramagnetic metal ion/L to 1000 mmol paramagnetic metal ion/L (inclusive), particularly in a range of 60 to 750 mmol paramagnetic metal ion/L (inclusive), particularly in a range of 70 to 700 mmol paramagnetic metal ion/L (inclusive), particularly in a range of 80 to 650 mmol paramagnetic metal ion/L (inclusive), particularly a the range of 90 to 600 mmol paramagnetic metal ion/L (inclusive), particularly in a range of 100 to 500 mmol paramagnetic metal ion/L (inclusive), particularly in a range of 150 to 450 mmol paramagnetic metal ion/L (inclusive), more particularly in a range of 200 to 400 mmol paramagnetic metal ion/L (inclusive), and even more particularly in a range of 250 to 350 mmol paramagnetic metal ion/L (inclusive).

In accordance with a fifth embodiment of the second aspect, the present disclosure covers a process for preparing a liquid pharmaceutical formulation, wherein the step of dissolving the DO3A-derived tetra-chelate of formula (I) comprises:
dissolving the Gd₄-DO3A-derived tetra-chelate of formula (I) as defined supra, in a pharmaceutically acceptable solvent or an aqueous buffer to provide a first solution, wherein the Gd₄-DO3A-derived tetra-chelate of formula (I) is dissolved in sufficient amount to produce a liquid pharmaceutical formulation having a concentration of the Gd₄-DO3A-derived tetra-chelate of formula (I) in a range of 60 to 750 mmol Gd³⁺/L (inclusive), particularly in a range of 70 to 700 mmol Gd³⁺/L (inclusive), particularly in a range of 80 to 650 mmol Gd³⁺/L (inclusive), particularly in a range of 90 to 600 mmol Gd³⁺/L (inclusive), particularly in a range of 100 to 500 mmol Gd³⁺/L (inclusive), particularly in a range of 150 to 450 mmol Gd³⁺/L (inclusive), more particularly in a range of 200 to 400 mmol Gd³⁺/L (inclusive), and even more particularly in a range of 250 to 350 mmol Gd³⁺/L (inclusive).

In accordance with a sixth embodiment of the second aspect, the present disclosure covers a process for preparing a formulation according to the present disclosure, wherein the process comprises:
dissolving an amount of a compound capable of forming a complex with free paramagnetic metal ion M in a range of 0.002% to 5% mol/mol (inclusive) with reference to the total concentration of the paramagnetic metal ion in the formulation in a pharmaceutically acceptable solvent or an aqueous buffer solution to provide a first solution;
dissolving a Gd₄-DO3A-derived tetra-chelate of formula (I) as defined supra, in sufficient amount to produce a final solution having a liquid pharmaceutical formulation having a concentration of the DO3A-derived tetra-chelate of formula (I) in the range of 1 mmol Gd³⁺/L to 1000 mmol Gd³⁺/L (inclusive), particularly in the range of 60 to 750 mmol Gd³⁺/L (inclusive), particularly in the range of 70 to 700 mmol Gd³⁺/L (inclusive), particularly in the range of 80 to 650 mmol Gd³⁺/L (inclusive), particularly in the range of 90 to 600 mmol Gd³⁺/L (inclusive), particularly in the range of 100 to 500 mmol Gd³⁺/L (inclusive), particularly in the range of 150 to 450 mmol Gd³⁺/L (inclusive), more particularly in the range of 200 to 400 mmol Gd³⁺/L (inclusive), and even more particularly in the range of 250 to 350 mmol Gd³*/L (inclusive).

In accordance with a seventh embodiment of the second aspect, the present disclosure covers a process for preparing a formulation according to the present disclosure, wherein the process comprises:
dissolving an amount of Ca-BT-DO3A in the range of 0.002% to 5% mol/mol (inclusive) with reference to the total concentration of the paramagnetic metal ion in the formulation in a pharmaceutically acceptable solvent or an aqueous buffer solution to provide a first solution;
dissolving a Gd₄-DO3A-derived tetra-chelate of formula (I) as defined supra, in sufficient amount to produce a final solution having a liquid pharmaceutical formulation having a concentration of the Gd₄-DO3A-derived tetra-chelate of formula (I) in the range of 1 mmol Gd³⁺/L to 1000 mmol Gd³⁺/L (inclusive), particularly in the range of 60 to 750 mmol Gd³⁺/L (inclusive), particularly in the range of 70 to 700 mmol Gd³⁺/L (inclusive), particularly in the range of 80 to 650 mmol Gd³⁺/L (inclusive), particularly in the range of 90 to 600 mmol Gd³⁺/L (inclusive), particularly in the range of 100 to 500 mmol Gd³⁺/L (inclusive), particularly in the range of 150 to 450 mmol Gd³⁺/L (inclusive), more particularly in the range of 200 to 400 mmol Gd³⁺/L (inclusive), and even more particularly in the range of 250 to 350 mmol Gd³⁺/L (inclusive).

In accordance with an eighth embodiment of the second aspect, the present disclosure covers a process for preparing a liquid pharmaceutical formulation, wherein the step of dissolving the DO3A-derived tetra-chelate of formula (I) comprises:
dissolving a Gd₄-DO3A-derived tetra-chelate of formula (I-a) as defined supra, in the pharmaceutically acceptable solvent or an aqueous buffer to provide a first solution, wherein the Gd₄-DO3A-derived tetra-chelate of formula (I-a) is dissolved in sufficient amount to produce a liquid pharmaceutical formulation having a concentration of the Gd₄-DO3A-derived tetra-chelate of formula (I-a) in the range of 60 to 750 mmol Gd³⁺/L (inclusive), particularly in a range of 70 to 700 mmol Gd³⁺/L (inclusive), particularly in a range of 80 to 650 mmol Gd³⁺/L (inclusive), particularly in a range of 90 to 600 mmol Gd³⁺/L (inclusive), particularly in a range of 100 to 500 mmol Gd³⁺/L (inclusive), particularly in a range of 150 to 450 mmol Gd³⁺/L (inclusive), more particularly in a range of 200 to 400 mmol Gd³⁺/L (inclusive), and even more particularly in a range of 250 to 350 mmol Gd³⁺/L (inclusive).

In accordance with a ninth embodiment of the second aspect, the present disclosure covers a process for preparing a formulation according to the present disclosure, wherein the process comprises:
dissolving an amount of a compound capable of forming a complex with free paramagnetic metal ion M in a range of 0.002% to 5% mol/mol (inclusive) with reference to the total concentration of the paramagnetic metal ion in the formulation in the pharmaceutically acceptable solvent or an aqueous buffer solution to provide a first solution;
dissolving a Gd₄-DO3A-derived tetra-chelate of formula (I-a) as defined supra, in the first solution in sufficient amount to produce a final solution having a liquid pharmaceutical formulation having a concentration of the Gd₄-DO3A-derived tetra-chelate of formula (I-a) in a range of 1 mmol Gd³⁺/L to 1000 mmol Gd³⁺/L (inclusive), particularly in a range of 60 to 750 mmol Gd³⁺/L (inclusive), particularly in a range of 70 to 700 mmol Gd³⁺/L (inclusive), particularly in a range of 80 to 650 mmol Gd³⁺/L (inclusive), particularly in a range of 90 to 600 mmol Gd³⁺/L (inclusive), particularly in a range of 100 to 500 mmol Gd³⁺/L (inclusive), particularly in a range of 150 to 450 mmol Gd³⁺/L (inclusive), more particularly in a range of 200 to 400 mmol Gd³⁺/L (inclusive), and even more particularly in a range of 250 to 350 mmol Gd³⁺/L (inclusive).

In accordance with a tenth embodiment of the second aspect, the present disclosure covers a process for preparing a formulation according to the present disclosure, wherein the process comprises:
dissolving an amount of Ca-BT-DO3A in a range of 0.002% to 5% mol/mol (inclusive) with reference to the total concentration of the paramagnetic metal ion in the formulation in the pharmaceutically acceptable solvent or an aqueous buffer solution to provide a first solution;
dissolving a Gd₄-DO3A-derived tetra-chelate of formula (I-a) as defined supra, in the first solution in sufficient amount to produce a final solution having a liquid pharmaceutical formulation having a concentration of the Gd₄-DO3A-derived tetra-chelate of formula (I-a) in a range of 1 mmol Gd³⁺/L to 1000 mmol Gd³⁺/L (inclusive), particularly in a range of 60 to 750 mmol Gd³⁺/L (inclusive), particularly in a range of 70 to 700 mmol Gd³⁺/L (inclusive), particularly in a range of 80 to 650 mmol Gd³⁺/L (inclusive), particularly in a range of 90 to 600 mmol Gd³⁺/L (inclusive), particularly in a range of 100 to 500 mmol Gd³⁺/L (inclusive), particularly in a range of 150 to 450 mmol Gd³⁺/L (inclusive), more particularly in a range of 200 to 400 mmol Gd³⁺/L (inclusive), and even more particularly in a range of 250 to 350 mmol Gd³⁺/L (inclusive).

In accordance with a eleventh embodiment of the second aspect, the present disclosure covers a process for preparing a formulation according to the present disclosure, wherein the process further comprises:
adjusting the pH of the contrast solution to a pH in the range of 4.5 to 8.5 (inclusive), particularly in the range of 6.6 to 8.0, (inclusive), more particularly in the range of 6.9 to 7.9 (inclusive), more particularly in the range of 7.2 to 7.6, more particularly adjusting the pH to 7.4.

In accordance with a twelfth embodiment of the second aspect, the process comprises the steps a) and d), and optionally any of the steps b), c), e), f), g) and h) or combinations thereof, said steps being as previously defined.

In accordance with a third aspect, the present disclosure covers a liquid pharmaceutical formulation obtained according to the process for preparing a formulation according to the various embodiments of the present disclosure.

### Use of the Formulations and Contrast Media

In accordance with a fourth aspect, the present disclosure covers the use of a formulation according to the present disclosure for medical imaging, or for diagnostic monitoring of the efficacy of a therapeutic treatment, comprising the administration of a pharmaceutically acceptable amount of a pharmaceutical formulation as described above.

Embodiments of the present disclosure thus relate to a contrast medium for medical imaging, comprising such a liquid pharmaceutical formulation.

In accordance with a second embodiment of the fourth aspect, the present disclosure covers the use of a formulation according to the present disclosure or the contrast media previously described for contrast-enhanced MRI sequences for all body regions. Applications for the formulation according to the present disclosure include cardiovascular, oncological and inflammatory indications for different body regions.

In accordance with a third embodiment of the fourth aspect, the present disclosure covers the use of a formulation according to the present disclosure or the contrast media previously described for the detection and characterization of CNS lesions, lesions of liver and abdomen, lesion in kidney and pelvis, in MR-angiography, as well as for indications in other organs/regions (i.e. tongue, head and neck, cardiovascular system, breast, chest, extremities, joints).

In accordance with a fourth embodiment of the fourth aspect, the present disclosure covers the use of the formulations or the contrast media previously described in the diagnosis of diseases, in particular cancerous, inflammatory, neurological or vascular diseases.

The various embodiments of the present disclosure also relate to said formulations or said contrast media which were previously described, for the use thereof in an imaging method, in particular a method as described below.

In accordance with a fifth aspect, the present disclosure relates to a method for imaging the whole body of an individual, or imaging a part of the body, comprising a step of obtaining one or more images of the whole body or of a part of the body of an individual by means of a medical imaging technique, in which the whole body or the part of the body of the individual comprises the formulation as described supra, in which the image contrast of one or more images is associated with the presence of the DO3A-derived tetra-chelate of general formula (I).

According to another embodiment, the imaging method according to the present disclosure comprises a prior step of injection or of administration of the formulation of the contrast medium to the individual, preferably a parenteral administration, preferably an intravenous, an intra-arterial or an intra-articular injection.

In the medical imaging methods defined above, the images are preferably obtained by Magnetic Resonance Imaging (MRI).

For a diagnosis by MRI, the intravenous administration by injection is typically carried out at a dose in the range of 0.01 to 0.3 mmol Gd/kg body weight (inclusive). The pharmaceutically acceptable dose will depend on the route of administration, and also on the patient and in particular on the nature of the disorder to be studied.

For an intravenous injection and observation by MRI, the concentration of the formulation is typically in the range of 1 to 1000 mmol Gd/L (inclusive), and the dose administered to the patient according to his or her weight will be, as appropriate, in the range of 0.01 to 0.3 mmol Gd/kg body weight (inclusive) and preferably in the range of 0.01 to 0.1 mmol Gd/kg body weight.

Among the advantageous diagnostic indications, mention will be made of the indications already clinically used, and the indications for which the diagnostic outcome are improved by the use of contrast agents.

In accordance with a sixth aspect, the present disclosure covers DO3A-derived tetra-chelates with sub-stoichiometric amounts of gadolinium ions (Gd³⁺), which are selected from the group consisting of:

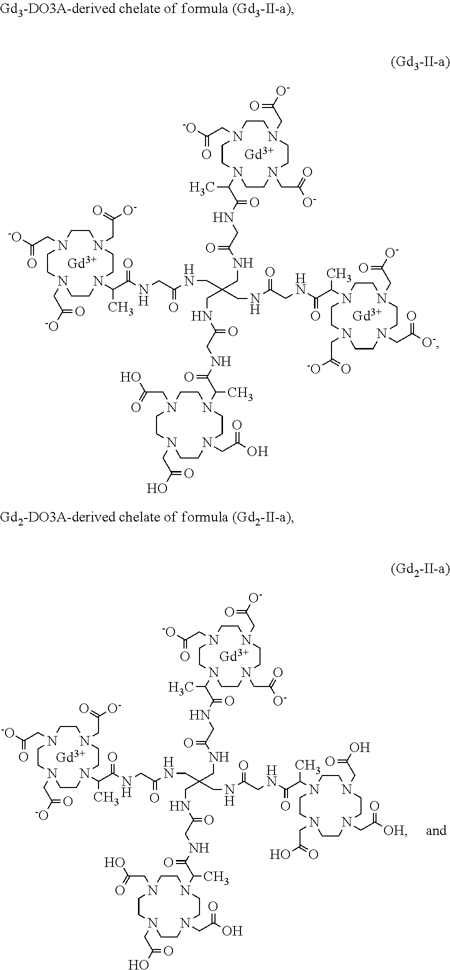

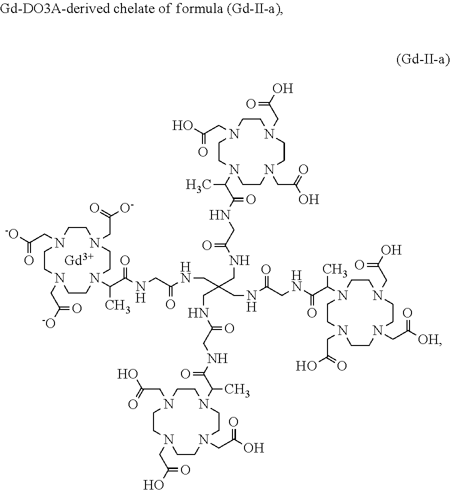

or a stereoisomer, a tautomer or a salt thereof, or a mixture of same.

In accordance with a second embodiment of the sixth aspect, the present disclosure covers DO3A-derived tetra-chelates with sub-stoichiometric amounts of gadolinium ions (Gd³⁺), which are selected from the group consisting of:

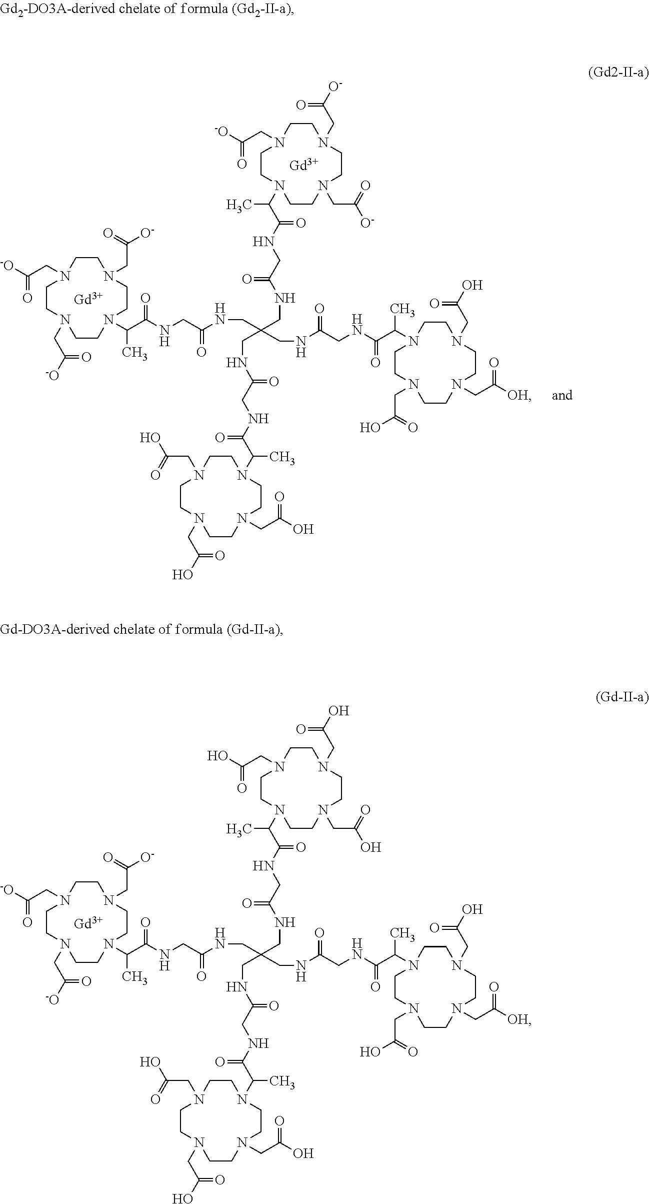

or a stereoisomer, a tautomer or a salt thereof, or a mixture of same.

In accordance with a variant of the sixth aspect, the present disclosure covers DO3A-derived tetra-chelates with sub-stoichiometric amounts of gadolinium ions (Gd³⁺), which are selected from the group consisting of:

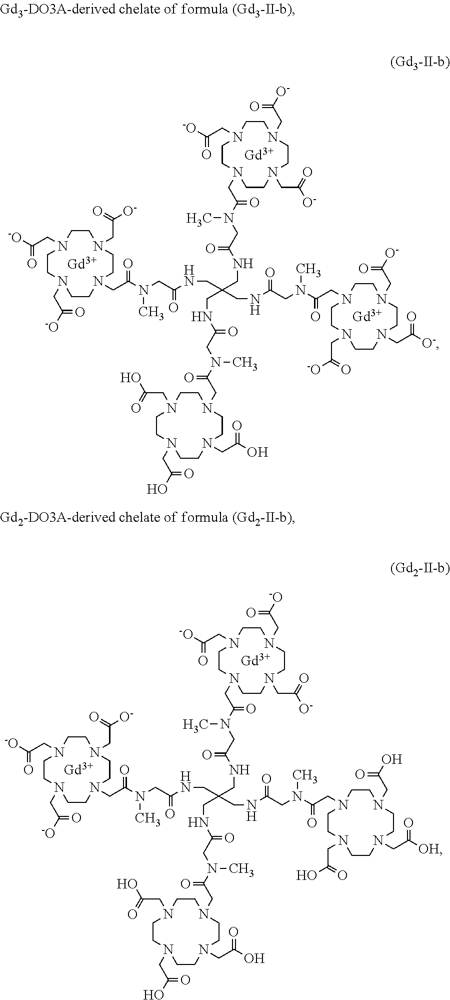

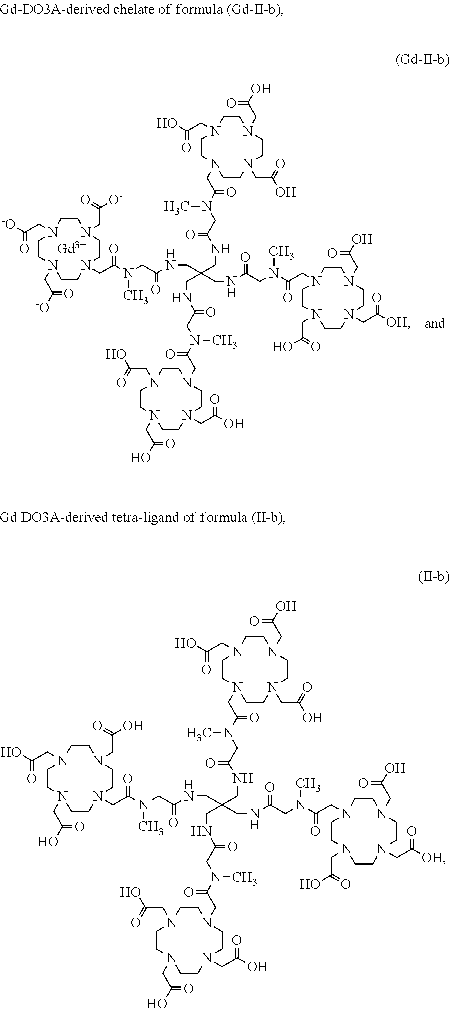

or a stereoisomer, a tautomer or a salt thereof, or a mixture of same.

In accordance with another variant of the sixth aspect, the present disclosure covers DO3A-derived tetra-chelates with sub-stoichiometric amounts of gadolinium ions (Gd³⁺), which are selected from the group consisting of:

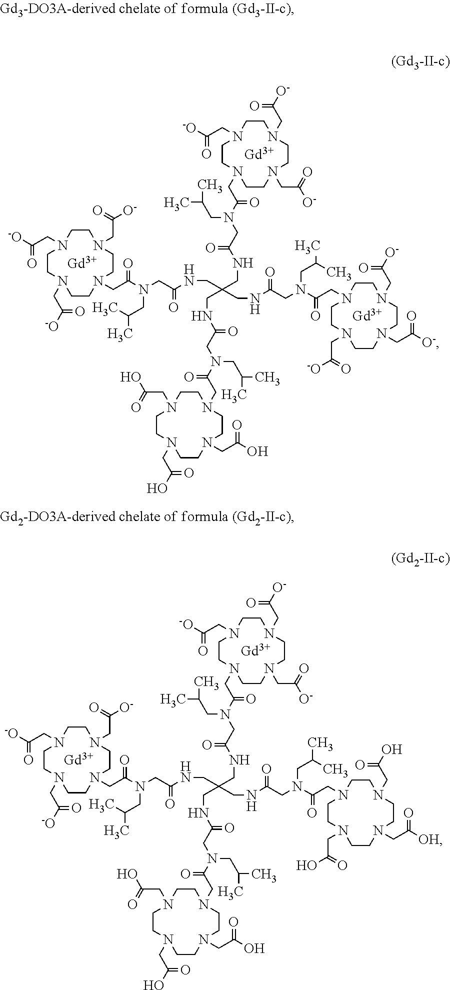

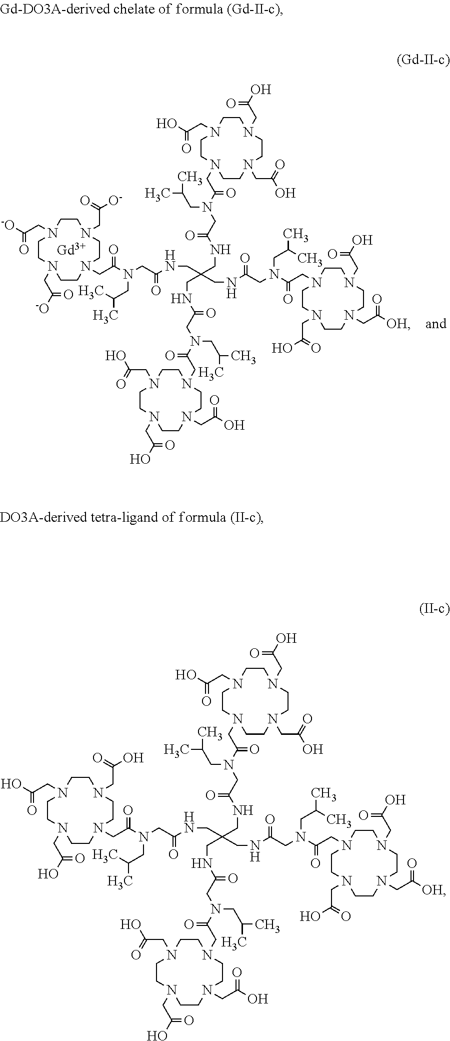

or a stereoisomer, a tautomer or a salt thereof, or a mixture of same.

In accordance with a seventh aspect, the present disclosure covers the use of DO3A-derived tetra-chelates with sub-stoichiometric amounts of gadolinium ions (Gd³⁺), which are selected from the group consisting of:

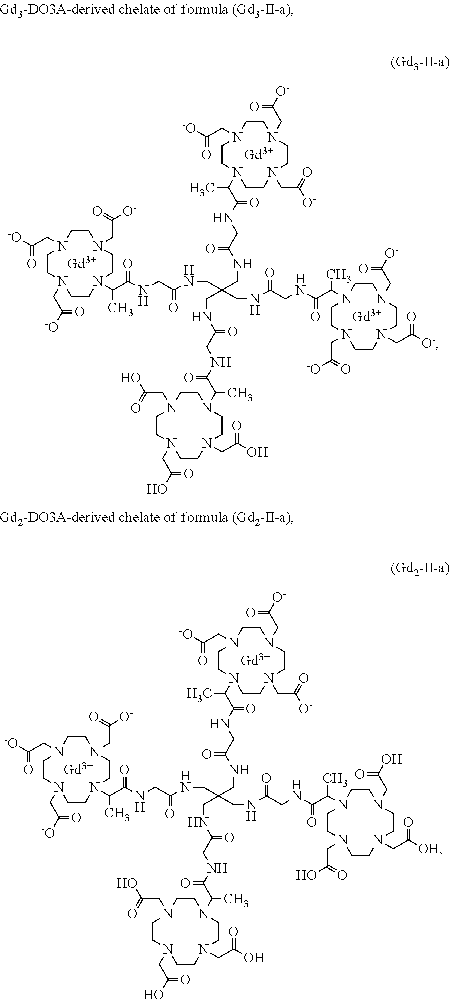

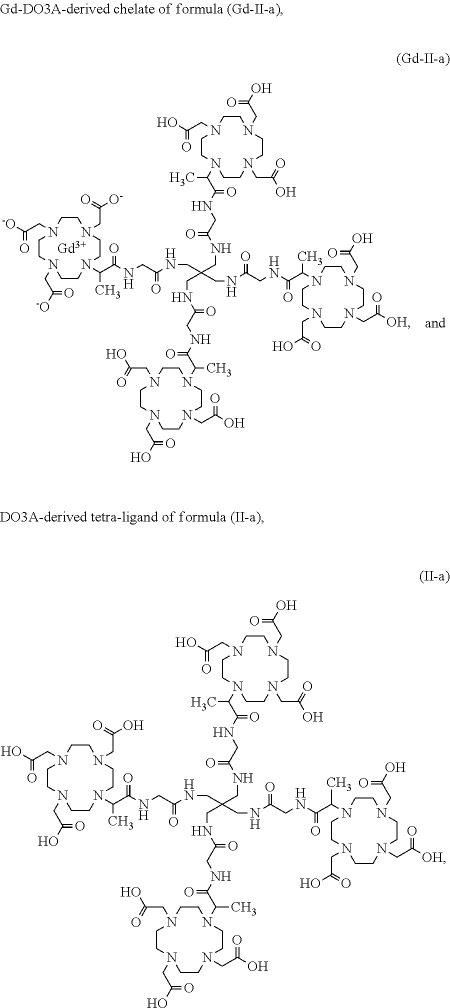

or a stereoisomer, a tautomer or a salt thereof, or a mixture of same,
for the manufacture of contrast agents for magnetic resonance imaging.

In accordance with a second embodiment of the seventh aspect, the present disclosure covers the use of DO3A-derived tetra-chelates with sub-stoichiometric amounts of gadolinium ions (Gd³⁺) which are selected from the group consisting of:

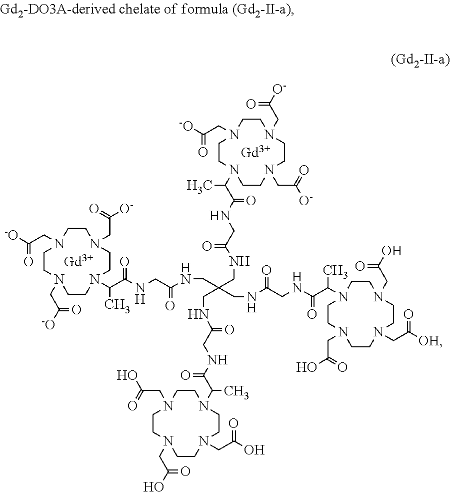

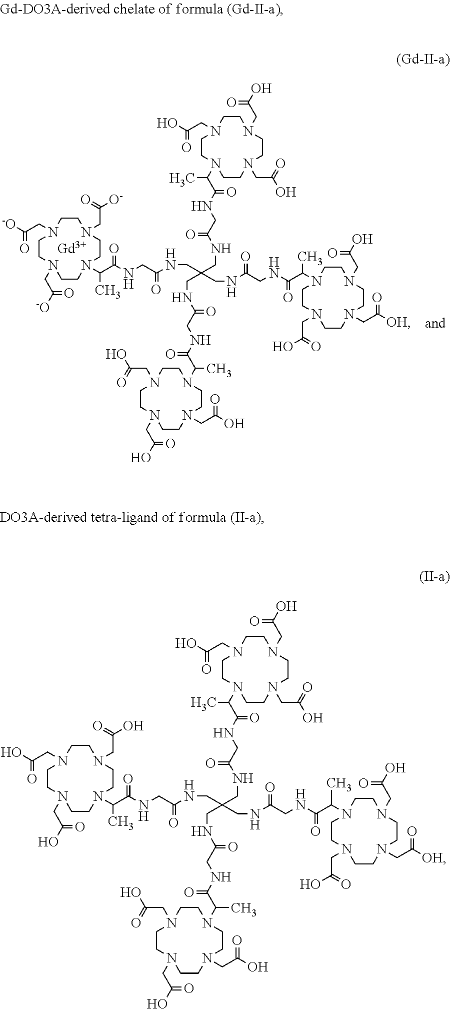

or a stereoisomer, a tautomer or a salt thereof, or a mixture of same,
for the manufacture of contrast agents for magnetic resonance imaging.

In accordance with a variant of the seventh aspect, the present disclosure covers the use of DO3A-derived tetra-chelates with sub-stoichiometric amounts of gadolinium ions (Gd³⁺), which are selected from the group consisting of:

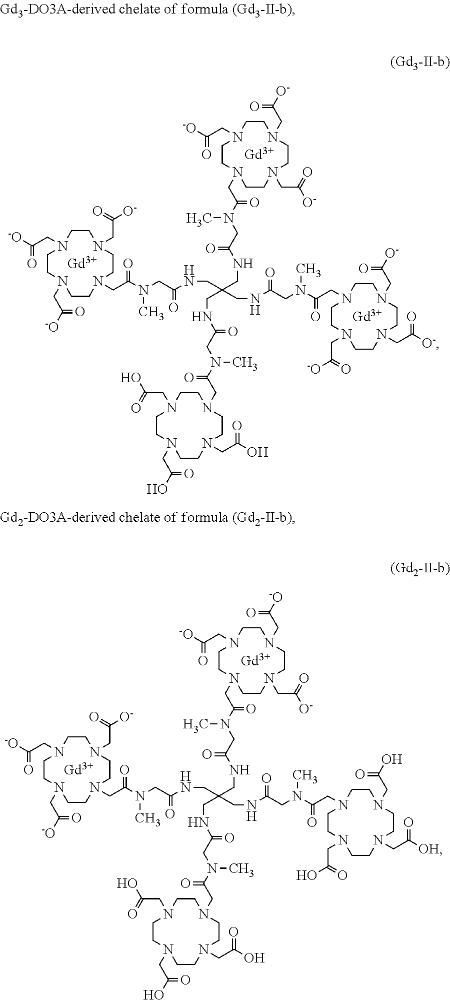

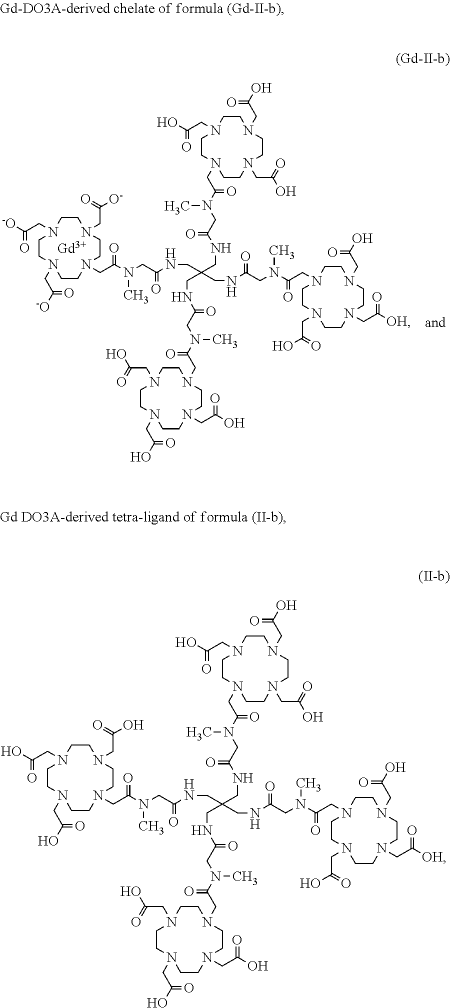

or a stereoisomer, a tautomer or a salt thereof, or a mixture of same,
for the manufacture of contrast agents for magnetic resonance imaging.

In accordance with another variant of the seventh aspect, the present disclosure covers the use of DO3A-derived tetra-chelates with sub-stoichiometric amounts of gadolinium ions (Gd³⁺), which are selected from the group consisting of:

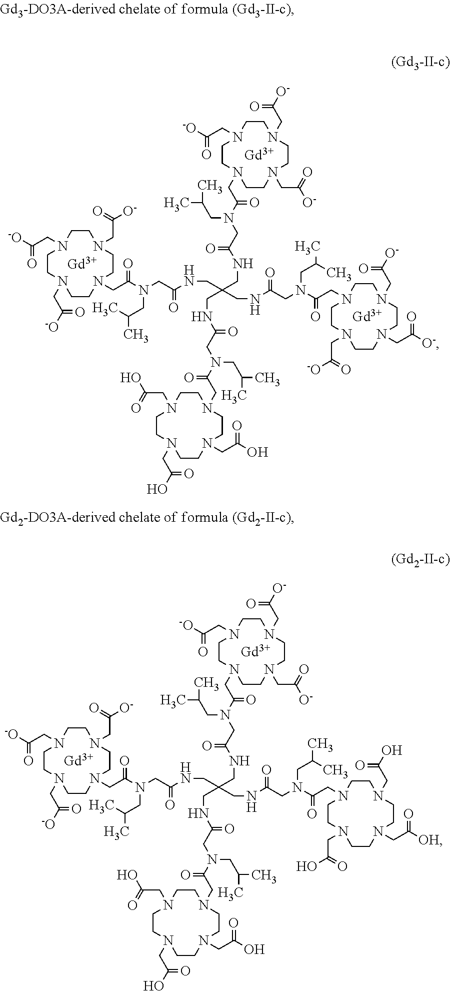

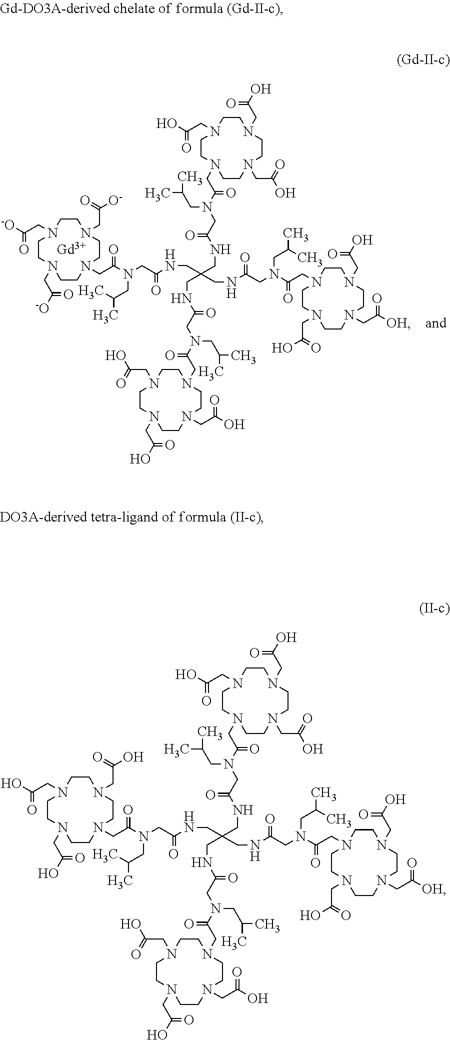

or a stereoisomer, a tautomer or a salt thereof, or a mixture of same,
for the manufacture of contrast agents for magnetic resonance imaging.

The various embodiments of the present disclosure will be illustrated by means of the non-limiting examples which follow.

### EXPERIMENTAL SECTION

### Example 1—Production of a Liquid Pharmaceutical Formulation Containing the Gd₄-DO3A-Derived Tetra-Chelate of Formula (I-a) and Ca-BT-DO3A (Calcobutrol)

The process for producing a liquid pharmaceutical formulation was carried out according to the following steps:
a) A manufacturing vessel was charged with 720.5 g water for injections and 1.217 g trometamol was dissolved while stirring. The pH of the solution obtained in step a) was adjusted to a pH of 7.6 to 8.2 by decreasing the pH by adding a 0.1 N solution of hydrochloric acid.
b) 0.147 g [i.e. 0.1% mol/mol related to the total Gd concentration] of Ca-BT-DO3A was added and dissolved while stirring.
c) 193.4 g (i.e. 0.075 M) of the Gd₄-DO3A-derived tetra-chelate of formula (I-a) was added to the solution obtained in step b) and dissolved while stirring.
d) 4.4 g of sodium chloride was added to the solution obtained in step c) and dissolved while stirring.
e) The pH of the solution obtained in step d) was adjusted to a pH of 7.2 to 7.6 by decreasing the pH by adding a 0.1 N solution of hydrochloric acid. The density of the solution was adjusted to a target value of 1.0998 g/mL by adding water. The solution was then filtered through a sterile filter with 0.2 μm pore size and placed in a container, which was subjected to sterilization at 121° C. for at least 15 minutes to yield the liquid pharmaceutical formulation.

By the procedure described above, the following formulation was obtained:

TABLE 2
Proportions in the formulation of Gd₄-DO3A-derived
tetra-chelate of formula (I-a)

| Ingredients | Proportions in the formulation | |
|---|---|---|
| Gd₄-DO3A-derived tetra-chelate | 193.4 g (0.3 mol Gd/L) | |
| of formula (I-a) | | |
| Ca-BT-DO3A | 0.147 g (0.3 mmol/L, i.e. | |
| | 0.1% mol/mol vs Gd) | |
| trometamol | 10 | mmol/L |
| sodium chloride | 4.4 | g |

| | |
|---|---|
| HCl | quantum satis, pH 7.4 ± 0.5 |
| water for injection | quantum satis 1 L |
| | (final volume of formulation = 1 L) |

The assaying of free gadolinium was carried out by colorimetry with Xylenol Orange. Xylenol Orange forms, with the free gadolinium, a colored complex having a specific absorbance (Barge et. al. Contrast Media & Molecular Imaging, 2006; 1; 184). Testing was conducted in comparison to a solution of gadolinium sulfate containing 2 ppm (m/v) of gadolinium. The final formulation contained less than or equal to 2 ppm (m/v) free gadolinium.

### Example 2—Stability and Osmolality

Stability studies and measurements of osmolality were carried out with the formulation obtained as described in example 1.

Measurements, over the course of time, of the concentration of the Gd₄-DO3A-derived tetra-chelate of formula (I-a), free gadolinium and osmolality were carried out.

TABLE 3
Stability and Osmolality

| | Storage | | | | |
|---|---|---|---|---|---|
| | duration | 2-8° C./ | 25° C./ | 30° C./ | 40° C./ |
| | [months] | — | 60% RH | 75% RH | 75% RH |
| concentration of | Initial | 187.7 | 187.7 | 187.7 | 187.7 |
| Gd4-DO3A-derived | 1 | Not tested | Not tested | Not tested | 187.7 |
| tetra-chelate of | 3 | Not tested | Not tested | Not tested | 193.0 |
| formula (I-a) | 6 | 193.2 | 194.5 | Not tested | 194.9 |
| [g/L] | 9 | Not tested | Not tested | 192.1 | Not tested |
| | 12 | Not tested | Not tested | 194.2 | Not tested |
| | 18 | Not tested | Not tested | 192.7 | Not tested |
| amount of free | Initial | ≤2 | ≤2 | ≤2 | ≤2 |
| gadolinium | 1 | Not tested | Not tested | Not tested | ≤2 |
| [ppm (m/v)] | 3 | Not tested | Not tested | Not tested | ≤2 |
| | 6 | ≤2 | ≤2 | Not tested | ≤2 |
| | 9 | Not tested | Not tested | ≤2 | Not tested |
| | 12 | Not tested | Not tested | ≤2 | Not tested |
| | 18 | Not tested | Not tested | ≤2 | Not tested |
| osmolality of | Initial | 294 | 294 | 294 | 294 |
| Gd₄-DO3A-derived | 1 | Not tested | Not tested | Not tested | 288 |
| tetra-chelate of | 3 | Not tested | Not tested | Not tested | 283 |
| formula (I-a) | 6 | 283 | 284 | Not tested | 283 |
| [mOsm/kg] | 9 | Not tested | Not tested | 282 | Not tested |
| | 12 | Not tested | 296 | 306 | Not tested |
| | 18 | Not tested | Not tested | 280 | Not tested |

Quantification of the Gd₄-DO3A-derived tetra-chelate of formula (I-a) was conducted by HPLC-UV against external standard solutions. Osmolality was determined using an automated vapor pressure osmometer.

The assaying of free gadolinium was carried out by colorimetry with Xylenol Orange. Xylenol Orange forms, with the free gadolinium, a colored complex having a specific absorbance (Barge et. al. Contrast Media & Molecular Imaging, 2006; 1; 184). Testing was conducted in comparison to a solution of gadolinium sulfate containing 2 ppm (m/v) of gadolinium.

The amount of Gd₄-DO3A-derived tetra-chelate of formula (I-a) remained stable after six months at 25° C. (long term stability) and also after six months at 40° C. (accelerated storage conditions). Free gadolinium was lower than or equal to 2 ppm (m/v) in the formulation. The formulation was isotonic with blood plasma.

### Example 3—Viscosity

Viscosity measurements were carried out with the formulation obtained as described in example 1, at 20° C. and 37° C.

TABLE 4
Viscosity

| | Viscosity | Viscosity |
|---|---|---|
| Product | 20° C. [mPas] | 37° C. [mPas] |
| Formulation containing the Gd₄-DO3A- | 1.87 | 1.22 |
| derived tetra-chelate of formula (I-a), | | |
| as described in example 1 | | |
| 0.9% (m/v) sodium chloride | 1.03 | 0.74 |
| Gadovist ® 1.0 | 8.86* | 4.96* |

*Fachinformation des Arzneimittel-Kompendium der Schweiz ®

Viscosities were determined using a microfluidic viscosimeter (m-VROC, RheoSense). The viscosities were only slightly higher than for isotonic sodium chloride solution but significantly lower than Gadovist® 1.0 and can be considered low.

### Example 4—Synthesis of DO3A-Derived Tetra-Ligand of Formula (II-a), Gd-DO3A-Derived Chelate (Gd-II-a), Gd₂-DO3A-Derived Chelate (Gd₂-II-a)

### [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetic acid (II-a)

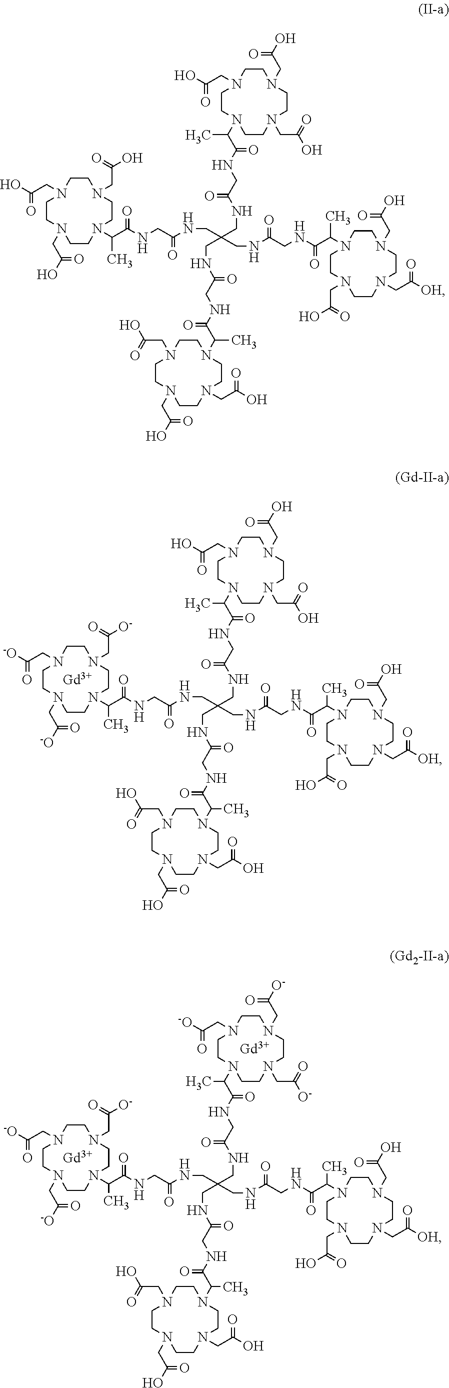

A solution of Gd₄-DO3A-derived tetra-chelate of formula (I-a), tetragadolinium [4,10-bis(carboxylatomethyl)-7-13.6.12,15-tetraoxo-16-[4,7,10-tris(carboxylatomethyl)-1.4.7,10-tetra-azacyclododecan-1-yl]-9,9-bis({[({2-[4,7,10-tris(carboxylatomethyl)-1,4,7,10-tetraazacyclo-dodecan-1-yl]propanoyl}amino)acetyl]amino}methyl)-4,7,11,14-tetraazaheptadecan-2-yl}-1,4,7,10-tetraazacyclododecan-1-yl]acetate (WO 2016193190, Example 3; 1.00 eq., 5.60 g, 2.12 mmol) in water (470 mL) was treated with oxalic acid dihydrate (16.0 eq., 4.29 g, 34.0 mmol) and stirred at 100° C. for 6 hours. The cooled reaction mixture was filtrated (Microfilter PTFE 1.2 μm) and lyophilized. The obtained crude material was dissolved in water (200 mL) and the pH adjusted to 4.5 by addition of aqueous sodium hydroxide solution (2 M). The obtained solution was ultrafiltrated with water (18×100 mL) using an 1 kDa membrane and the final retentate was lyophilized yielding 2.98 g solid white powder which was analyzed using ¹H-NMR and HPLC.

### ¹H-NMR:

(400 MHz, D₂O): δ [ppm]: 1.18-1.20 (m, 12H), 2.60-2.75 (m, 7H), 2.87-3.54 (m, 85H), 3.67-3.95 (m, 31H), 4.03 (q, 5H).

### HPLC:

Instrument: Agilent 1290 HPLC-ESI-MS G6130; column: Hypercarb (Thermo) 5 μm, 100×4.6 mm; Eluent A: water+0.1% formic acid, eluent B: acetonitrile+0.1% formic acid; gradient: 0-7 min 0-50% B, 7-8 min 100% B; flow rate 1 mL/min; temperature: 60° C.; injection: 20 μL; DAD scan: 200-300 nm; ESI-MS.

TABLE 5
HPLC

| | | | | Amount | | |
|---|---|---|---|---|---|---|
| | | Relative | | Gd-free | | |
| | Retention | amount | Amount | DO3A | Molecular | |
| | Time | [% peak | component | moieties | weight | MS |
| Compound | [min] | area] | [g] | [mmol] | [g/mol] | (ES⁺ and ES⁻) |
| II-a | 4.0 | 0.596 | 1.77 | 5.41 | 1961 | m/z (z = 2) = +982 |
| | | | | | | (M + 2H)²⁺; |
| | | | | | | m/z (z = 3) = +655 |
| | | | | | | (M + 3H)³⁺ |
| Gd-II-a | 4.6 | 0.313 | 0.93 | 1.32 | 2116 | m/z (z = 3) = +706 |
| | | | | | | (M + 3H)³⁺; |
| | | | | | | m/z (z = −2) = −1057 |
| | | | | | | (M − 2H)²⁻ |
| Gd₂-II-a | 5.5 | 0.091 | 0.27 | 0.32 | 2271 | m/z (z = 3) = +758 |
| | | | | | | (M + 3H)³⁺; |
| | | | | | | m/z (z = −2) = −1134 |
| | | | | | | (M − 2H)²⁻ |
| total | — | 1.00 | 2.98 | 6.96 | — | — |

The amount of each component was calculated from the % peak area (% PA) and the total weight (w) of the compound: % PA*w.

The total molar amount of Gd free DO3A moieties in the compound was calculated for each component based on their number of Gd ions per tetramer (nGd), their amount (a) and molecular weight (mw): (4−nGd)*a/mw.

### Example 5—Preparation of a Formulation for Injection of DO3A-Derived Tetra-Chelate of Formula (I-a) with a Defined Excess of Free DO3A Moieties (Present in Sub-Stoichiometric Chelates), Using the Mixture of Compounds of Example 4

29.8 g of Gd₄-DO3A-derived tetra-chelate of formula (I-a), water content 4.5% w/w, were dissolved in 115 mL of 10 mM Tris-HCl buffer, pH 7.4 in water for injection. The pH of the solution was adjusted to 7.4 using dilute aqueous sodium hydroxide and hydrochloric acid. The concentration was measured with ICP-OES: 334 mmol Gd/L. The volume of the solution was determined by weighing and under consideration of the density of the solution (1.10 g/mL): 132 mL. The solution contains 44.1 mmol Gd.

0.018 g of the mixture of compounds described in Example 4, equivalent to 0.044 mmol of free DO3A moieties, was added (0.018 g*6.96 mmol/2.98 g=0.044 mmol). The volume was adjusted to 176 mL with Tris-HCL buffer, pH 7.4.

The osmolality of the formulation was measured and an appropriate amount of sodium chloride was added to obtain 312 mOsm/kg which is isotonic to human blood.

Finally the solution was filtered through 0.22 μm in glass bottles which were sealed and steam autoclaved. A final measurement of the Gd-concentration confirmed 252 mmol Gd/L.

As a result an isotonic injection with 252 mmol Gd/L and an excess of 0.1 mol % of free ligand based on the total concentration of Gd and on free DO3A moieties was prepared (0.044 mmol/44.1 mmol=0.1 mol %).

The viscosity of the injection, as determined with a rolling ball viscometer (Paar) at 37° C., was 1.2 mPas.

We claim:

1. A DO3A-derived tetra-chelate with sub-stoichiometric amounts of gadolinium ions (Gd³⁺) which comprises a structure selected from the group consisting of: 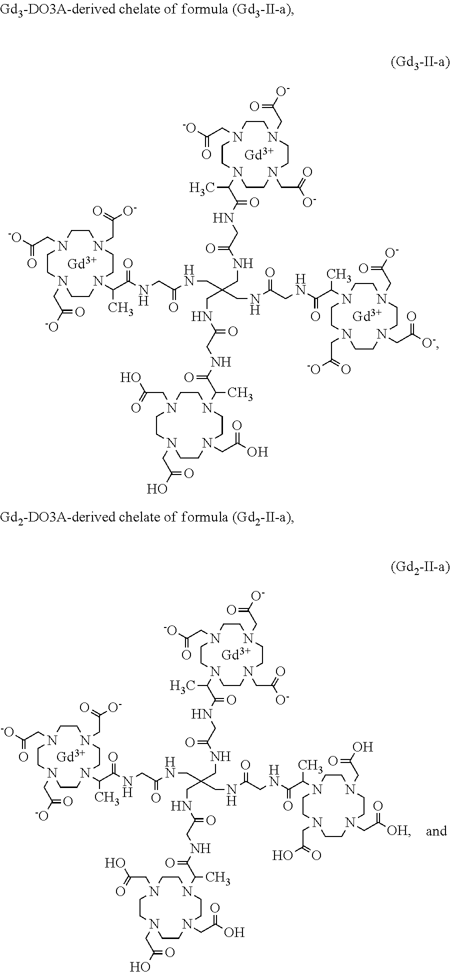 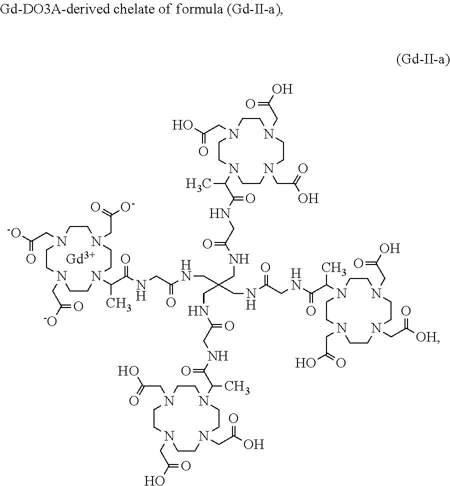
or a stereoisomer, a tautomer or a salt thereof, or a mixture of same.

2. The DO3A-derived tetra-chelate with sub-stoichiometric amounts of gadolinium ions (Gd³⁺) according to claim 1, wherein each chiral carbon atom in the DO3A-derived tetra-chelate has a stereochemistry that is either R or S.

3. The DO3A-derived tetra-chelate with sub-stoichiometric amounts of gadolinium ions (Gd³⁺) according to claim 1, wherein the DO3A-derived tetra-chelate is Trigadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate.

4. The DO3A-derived tetra-chelate with sub-stoichiometric amounts of gadolinium ions (Gd³⁺) according to claim 1, wherein the DO3A-derived tetra-chelate is Digadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate.

5. The DO3A-derived tetra-chelate with sub-stoichiometric amounts of gadolinium ions (Gd³⁺) according to claim 1, wherein the DO3A-derived tetra-chelate is Monogadolinium [4,10-bis(carboxylatomethyl)-7-{3,6,12,15-tetraoxo-16-[4,7,10-tris-(carboxylatomethyl)-1,4,7,10-tetraazacyclododecan-1-yl]-9,9-bis({[({2-[4,7,10-tris-(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate.

6. The DO3A-derived tetra-chelate with sub-stoichiometric amounts of gadolinium ions (Gd³⁺) according to claim 1, wherein the sub-stoichiometric amounts of gadolinium ions (Gd3+) comprises from 3.95 to 3.9996 moles of gadolinium ion relative to 1.000 moles of DO3A-derived tetral-ligand having the following structure

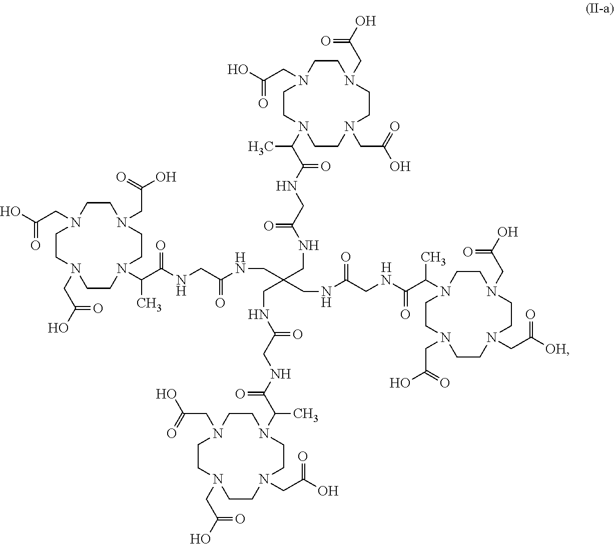

7. The DO3A-derived tetra-chelate with sub-stoichiometric amounts of gadolinium ions (Gd³⁺) according to claim 1, wherein the DO3A-derived tetra-chelate further comprises a counterion selected from the group consisting of Ca²⁺ ions, Na⁺ ions, Zn²⁺ ions, Mg²⁺ ions and/or meglumine ions.

8. A liquid pharmaceutical formulation comprising a DO3A-derived tetra-chelate of general formula (I): 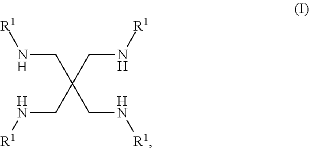
in which
R¹ represents a group selected from: 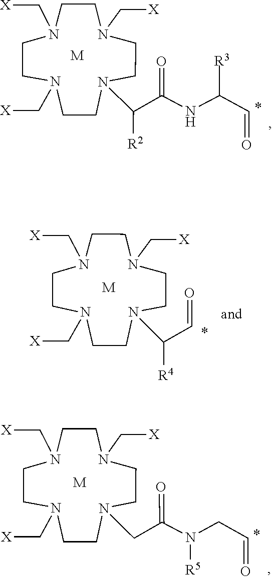
in which group * indicates the point of attachment of said group with the rest of the molecule,
R², R³, and R⁴ independently of each other represents a hydrogen atom or a group selected from:
C₁-C₆-alkyl, C₃-C₆-cycloalkyl, (C₁-C₂-alkoxy)-(C₂-C₃-alkyl)-, and phenyl,
wherein said C₁-C₆-alkyl group is optionally substituted, identically or differently, with a phenyl substituent, which phenyl substituent is optionally substituted, one, two, or three times, identically or differently, with a halogen atom or a group selected from:
C₁-C₃-alkyl, C₁-C₃-haloalkyl, and C₁-C₃-alkoxy, and
wherein said phenyl group is optionally substituted, one, two, or three times, identically or differently, with a halogen atom or a group selected from:
C₁-C₃-alkyl, C₁-C₃-haloalkyl, and C₁-C₃-alkoxy,
R⁵ represents a group selected from:
C₁-C₆-alkyl, C₃-C₆-cycloalkyl, (C₁-C₂-alkoxy)-(C₂-C₃-alkyl)-, and phenyl,
wherein said C₁-C₆-alkyl group is optionally substituted, identically or differently, with a phenyl substituent, which phenyl substituent is optionally substituted, one, two, or three times, identically or differently, with a halogen atom or a group selected from:
C₁-C₃-alkyl, C₁-C₃-haloalkyl, and C₁-C₃-alkoxy, and
wherein said phenyl group is optionally substituted, one, two, or three times, identically or differently, with a halogen atom or a group selected from:
C₁-C₃-alkyl, C₁-C₃-haloalkyl, and C₁-C₃-alkoxy,
X represents a group C(═O)O⁻, and
each M represents a Gd³⁺ ion,
or a stereoisomer, a tautomer, a salt thereof, or a mixture of same,
said formulation further comprising
a sub-stoichiometric chelate, as described herein, in a concentration fraction of 0.01 to 1.25 mol % [with reference to the total molar Gd-concentration], wherein the sub-stoichiometric chelate is selected from one or more of Gd₃-DO3A-derived chelate, Gd₂-DO3A-derived chelate, Gd-DO3A-derived chelate, and the DO3A-derived tetra-ligand, or mixtures thereof or stereoisomers, tautomers, salts thereof, or a mixture of any thereof,
wherein the one, two or three DO3A ligands of said sub-stoichiometric chelates, which are not complexed with Gd³⁺ ions,
and the four DO3A ligands of the DO3A-derived tetra-ligand, may be present as complexes or salts with Ca²⁺ ions, Na⁺ ions, Zn²⁺ ions, Mg²⁺ ions, or meglumine ions or as free carboxylic acids,
a pharmaceutically acceptable solvent, and
optionally comprising a buffer,
wherein the DO3A-derived tetra-chelate of general formula (I) has a concentration in the formulation in a range of 1 mmol Gd³⁺/L to 1000 mmol Gd³⁺/L (inclusive).

9. The liquid pharmaceutical formulation comprising a DO3A-derived tetra-chelate of claim 8, wherein the DO3A-derived tetra-chelated is selected from the group consisting of
Tetragadolinium [4,10-bis(carboxylatomethyl)-7-{(2R,16R)-3,6,12,15-tetraoxo-16-[4,7,10-tris-(carboxylatomethyl)-1,4,7,10-tetraazacyclododecan-1-yl]-9,9-bis({[({(2R)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate,
Tetragadolinium [4,10-bis(carboxylatomethyl)-7-{(2S,16S)-3,6,12,15-tetraoxo-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate,
Tetragadolinium [4,10-bis(carboxylatomethyl)-7-{(2R,16S)-3,6,12,15-tetraox9.o-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate,
Tetragadolinium [4,10-bis(carboxylatomethyl)-7-{(2R,16R)-3,6,12,15-tetraoxo-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, and
Tetragadolinium [4,10-bis(carboxylatomethyl)-7-{(2S,16R)-3,6,12,15-tetraoxo-16-[4,7,10-tris-(carboxylatomethyl)-1,4,7,10-tetraazacyclododecan-1-yl]-9,9-bis({[({(2R)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate,
or a tautomer or a salt thereof, or a mixture of same

10. A liquid pharmaceutical formulation comprising:
a DO3A-derived tetra-chelate selected from the group consisting of Tetragadolinium [4,10-bis(carboxylatomethyl)-7-{(2R,16R)-3,6,12,15-tetraoxo-16-[4,7,10-tris(carboxylatomethyl)-1,4,7,10-tetraazacyclododecan-1-yl]-9,9-bis({[({(2R)-2-[4,7,10-tris(carboxylatomethyl)-1,4,7,10-tetraazacyclododecan-1-yl]propanoyl}amino)acetyl]amino}methyl)-4,7,11,14-tetraazaheptadecan-2-yl}-1,4,7,10-tetraazacyclododecan-1-yl]acetate,
Tetragadolinium [4,10-bis(carboxylatomethyl)-7-{(2S,16S)-3,6,12,15-tetraoxo-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate,
Tetragadolinium [4,10-bis(carboxylatomethyl)-7-{(2R,16S)-3,6,12,15-tetraox9.o-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, Tetragadolinium [4,10-bis(carboxylatomethyl)-7-{(2R,16R)-3,6,12,15-tetraoxo-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, and
Tetragadolinium [4,10-bis(carboxylatomethyl)-7-{(2S,16R)-3,6,12,15-tetraoxo-16-[4,7,10-tris-(carboxylatomethyl)-1,4,7,10-tetraazacyclododecan-1-yl]-9,9-bis({[({(2R)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate,
or a tautomer or a salt thereof, or a mixture of same in a range of from 70 to 700 mmol of Gd³⁺ ion/L;
a compound capable of forming a chelate with a free gadolinium metal ion Gd³⁺ comprising Ca-BT-DO3A (Calcobutrol) in a range of 0.002% to 5% mol/mol (inclusive) measured as a proportion relative to a total Gd³⁺ concentration in the liquid pharmaceutical formulation;
a buffer in the form of 10.0 mmol/L Trometamol (TRIS, 2-amino-2-(hydroxymethyl)propane-1,3-diol);
an isotonicity agent in the form of NaCl;
a pH adjusting agent in the form of 0.1 N HCl (aq); and
a pharmaceutically acceptable solvent in the form of water.

11. The liquid pharmaceutical formulation of claim 10, having a concentration of a free paramagnetic metal ion M of less than or equal to 5 ppm (m/v).

12. The liquid pharmaceutical formulation of claim 10, wherein
the buffer in the form of 10.0 mmol/L Trometamol (TRIS, 2-amino-2-(hydroxymethyl)propane-1,3-diol) and a buffer pH adjusting agent is present in an amount sufficient to to provide a pH in the range of 7.6 to 8.2;
the isotonicity agent in the form of NaCl is present in an amount sufficient to provide an osmolality in a range of 200 to 400 mOsm/Kg (inclusive);
the pH adjusting agent in the form of 0.1 N HCl (aq.) is present in an amount sufficient to adjust a pH of the liquid pharmaceutical formulation to a pH in a range of 6.9 to 7.9; and
the pharmaceutically acceptable solvent in the form of water is present in an amount sufficient to provide a final concentration of the liquid pharmaceutical formulation having the range of 70 to 700 mmol of Gd³⁺ ion/L.

13. The liquid pharmaceutical formulation of claim 12, wherein the isotonicity agent in the form of NaCl is present in an amount sufficient to provide an osmolality in a range of 250 to 300 mOsm/Kg (inclusive).

14. A method for making a liquid pharmaceutical formulation, the method comprising:
providing a sufficient volume of a pharmaceutically acceptable solvent in the form of water;
dissolving Trometamol (TRIS, 2-amino-2-(hydroxymethyl)propane-1,3-diol) buffer in an amount to produce a 10.0 mmol/L buffered solution and adjusting the pH of the buffered solution to a pH in a range of 7.6 to 8.2 (inclusive) by addition of a 0.1 N solution of 0.1 N HCl (aq);
dissolving an amount of Ca-BT-DO3A (Calcobutrol) in the buffered solution to provide a 0.1% mol/mol concentration of the Ca-BT-DO3A related to a total Gd³⁺ concentration in the liquid pharmaceutical formulation;
dissolving an amount of a DO3A-derived tetra-chelate selected from the group consisting of Tetragadolinium [4,10-bis(carboxylatomethyl)-7-{(2R,16R)-3,6,12,15-tetraoxo-16-[4,7,10-tris(carboxylatomethyl)-1,4,7,10-tetraazacyclododecan-1-yl]-9,9-bis({[({(2R)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, Tetragadolinium [4,10-bis(carboxylatomethyl)-7-{(2S,16S)-3,6,12,15-tetraoxo-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, Tetragadolinium [4,10-bis(carboxylatomethyl)-7-{(2R,16S)-3,6,12,15-tetraox9.o-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, Tetragadolinium [4,10-bis(carboxylatomethyl)-7-{(2R,16R)-3,6,12,15-tetraoxo-16-[4,7,10-tris-(carboxylatomethyl)-1,4,7,10-tetraazacyclododecan-1-yl]-9,9-bis({[({(2S)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, and
Tetragadolinium [4,10-bis(carboxylatomethyl)-7-{(2S,16R)-3,6,12,15-tetraoxo-16-[4,7,10-tris-(carboxylatomethyl)-1,4,7,10-tetraazacyclododecan-1-yl]-9,9-bis({[({(2R)-2-[4,7,10-tris-(carboxylatomethyl)-1,4,7,10-tetraazacyclododecan-1-yl]propanoyl}amino)acetyl]-amino}methyl)-4,7,11,14-tetraazaheptadecan-2-yl}-1,4,7,10-tetraazacyclododecan-1-yl]acetate, or a tautomer or a salt thereof, or a mixture of same in an amount sufficient to provide from 70 to 700 mmol of Gd³⁺ ion/L in in the liquid pharmaceutical formulation;
dissolving sodium chloride as an isotonicity agent in an amount sufficient to provide an osmolality in a range of 250 to 350 mOsm/Kg (inclusive);
adjusting a pH of the liquid pharmaceutical formulation with a 0.1 N solution of 0.1 N HCl (aq) to a pH in a range of 6.9 to 7.9; (inclusive); and
adding any additional water to the liquid pharmaceutical formulation necessary to provide the Gd³⁺ concentration of from 70 to 700 mmol of Gd³⁺ ion/L to provide the liquid pharmaceutical formulation.

15. The method of claim 14, further comprising sterilizing the liquid pharmaceutical formulation by heating the liquid pharmaceutical formulation to 121° C. for at least 15 minutes.
